(19) United States
(12) Patent Application Publication
(10) Pub. No.: US 2026/0069862 A1
(43) Pub. Date: Mar. 12, 2026

(54) WEARABLE DEVICE AND AROUSAL STATE CONTROL SYSTEM

(71) Applicants: Ippei KIMURA, Osaka (JP); Koji IWASAKI, Kanagawa (JP); Yoshihiro SEKINE, Aichi (JP)

(72) Inventors: Ippei KIMURA, Osaka (JP); Koji IWASAKI, Kanagawa (JP); Yoshihiro SEKINE, Aichi (JP)

(21) Appl. No.: 19/132,888

(22) PCT Filed: Nov. 20, 2023
(86) PCT No.: PCT/IB2023/061667

§ 371 Date: May 27, 2025

(30) Foreign Application Priority Data
Nov. 30, 2022 (JP) 2022-192274
Oct. 5, 2023 (JP) 2023-173617

(51) Int. Cl. A61N 1/36; A61M 21/02; A61N 1/04

(52) U.S. Cl. CPC A61N 1/36031; A61M 21/02; A61N 1/0476; A61N 1/0484

(57) ABSTRACT

A wearable device to be worn by a user includes a body and a plurality of stimulators on the body to apply, based on biological information of the user, stimuli of different types or different strengths to the user.

### TECHNICAL FIELD

Embodiments of the present disclosure relate to a wearable device and an arousal state control system.

### BACKGROUND ART

The autonomic nervous system is a nervous system that regulates autonomic functions such as respiration, digestion, circulatory, and body temperature maintenance. The balance between sympathetic nerves and parasympathetic nerves maintains the autonomic nervous system. Sympathetic nerves become active during arousal, whereas parasympathetic nerves become active during relaxation. Although high levels of arousal increase concentration, an excessively high level of arousal reduces performance due to excessive stress. Such a relationship between arousal and performance is known as the Yerkes-Dotson law. According to the Yerkes-Dotson law, the level of arousal and performance exhibits an inverted U-shaped correlation in a graph with the horizontal axis representing the level of arousal and the vertical axis representing the performance. The graph has a range of levels of arousal suitable for maximizing performance, whereas the performance decreases outside the range.

A stress suppression system has been proposed that measures biological information such as the body temperature and the heart rate of a user to induce a healing effect by, for example, images, music, or tactile stimulation (see, for example, Patent Literature (PTL) 1). A neck cosmetic device has been proposed that provides electrical stimulation or iontophoresis to the neck to exert cosmetic effects (see, for example, PTL 2).

### CITATION LIST

### Patent Literature

[PTL 1]
Japanese Unexamined Patent Application Publication No. 2020-202971
[PTL 2]
Japanese Unexamined Patent Application Publication No. 2021-108906

### SUMMARY OF INVENTION

### Technical Problem

The inverted U-shaped correlation of the Yerkes-Dotson law varies depending on the difficulty of the work or tasks being addressed. For simple tasks, a certain degree of tension increases the productivity. For complex tasks, relaxing tension may improve efficiency. The appropriate level of arousal varies between individuals. To enhance intellectual productivity, an appropriate stimulus for arousal is to be applied in addition to a healing effect. To lead a user to an appropriate state of arousal during work, it is desirable to apply a stimulus suitable for the user with a wearable device that does not disturb the work.

It is an object of the present invention to provide a wearable device and an arousal state control system including the wearable device that can control the state of arousal as appropriate for each situation in which the wearable device is used.

### Solution to Problem

A wearable device to be worn by a user includes a body and a plurality of stimulators on the body to apply, based on biological information of the user, stimuli of different types or different strengths to the user.

An arousal state control system includes a sensor, a stimulation control unit, and a wearable device. The sensor acquires biological information of a user. The stimulation control unit determines a plurality of types of stimuli to control a state of arousal of the user based on the biological information. The wearable device applies the plurality of types of stimuli determined by the stimulation control unit to the user. The wearable device includes a body and a stimulator on the body to apply the plurality of types of stimuli.

### Advantageous Effects of Invention

A wearable device is achieved that appropriately controls the state of arousal of a user with a combination of stimuli of different types or different strengths.

### BRIEF DESCRIPTION OF DRAWINGS

A more complete appreciation of embodiments of the present disclosure and many of the attendant advantages and features thereof can be readily obtained and understood from the following detailed description with reference to the accompanying drawings.

FIG. 1 is a schematic diagram of an arousal state control system according to an embodiment of the present disclosure.

FIG. 2 is a schematic diagram illustrating a wearable device worn by a user, according to an embodiment of the present disclosure.

FIG. 3A is a diagram of a wearable device according to a first example of the present disclosure.

FIG. 3B is a diagram of a wearable device according to a second example of the present disclosure.

FIG. 3C is a diagram of a wearable device according to a third example of the present disclosure.

FIG. 4 is a block diagram illustrating a hardware configuration of an information processing device according to an embodiment of the present disclosure.

FIG. 5 is a functional block diagram of a stimulation control unit according to an embodiment of the present disclosure.

FIG. 6A is a flowchart of control executed by a stimulation control unit according to an embodiment of the present disclosure.

FIG. 6B is a continuation of the flowchart of FIG. 6A.

FIG. 7 is a table illustrating the result of evaluations of arousal state control.

FIGS. 8A to 8C are views of a wearable device, illustrating a first configuration example of stimulators included in the wearable device.

FIGS. 9A to 9C are views of a wearable device, illustrating a second configuration example of stimulators included in the wearable device.

FIGS. 10A to 10D are partial views of a wearable device, illustrating a third configuration example of stimulators included in the wearable device.

FIGS. 11A to 11C are partial views of a wearable device, illustrating a fourth configuration example of stimulators included in the wearable device.

FIGS. 12A to 12C are views of a wearable device, illustrating a fifth configuration example of stimulators included in the wearable device.

FIGS. 13A and 13B are views of a wearable device, illustrating a sixth configuration example of stimulators included in the wearable device.

FIG. 14 is a perspective view of a wearable device, illustrating a seventh configuration example of stimulators included in the wearable device.

FIG. 15 is a diagram illustrating the wearable device of FIG. 14 and stimulation components detached from a body of the wearable device of FIG. 14.

FIG. 16A is a diagram illustrating the first step of attaching a stimulation component to a wearable device according to an embodiment of the present disclosure.

FIG. 16B is a diagram illustrating the second step of attaching the stimulation component to the wearable device of FIG. 16A.

FIG. 16C is a diagram illustrating the third step of attaching the stimulation component to the wearable device of FIG. 16A.

FIG. 16D is a diagram illustrating the fourth step of attaching the stimulation component to the wearable device of FIG. 16A.

FIG. 17 is a diagram of a wearable device according to another example of the present disclosure.

FIG. 18 is a schematic diagram illustrating a configuration of a skin-contact side of the wearable device of FIG. 17.

FIG. 19 is a diagram of a wearable device according to yet another example of the present disclosure.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DESCRIPTION OF EMBODIMENTS

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise. For the sake of simplicity, like reference signs denote like elements such as parts and materials having the same functions, and redundant descriptions thereof are omitted unless otherwise required. The sizes and relative positions of the components illustrated in the drawings may be exaggerated to facilitate understanding of the invention. As used herein, the term “connected/coupled” includes both direct connections and connections in which there are one or more intermediate connecting elements.

In the embodiments of the present disclosure, appropriate tension is given to a user, in addition to a relaxing effect, based on biological information to lead the user to an appropriate state of arousal. To achieve this, the biological information of the user is analyzed to generate and apply different types of stimuli. For example, a first stimulus for increasing tension or arousal is combined with a second stimulus for reducing tension or arousal. The state of arousal of the user is controlled as appropriate by simultaneous or separate application of different types of stimuli, to enhance intellectual productivity.

A description is given of configurations of an arousal state control system and a wearable device according to an embodiment of the present disclosure.

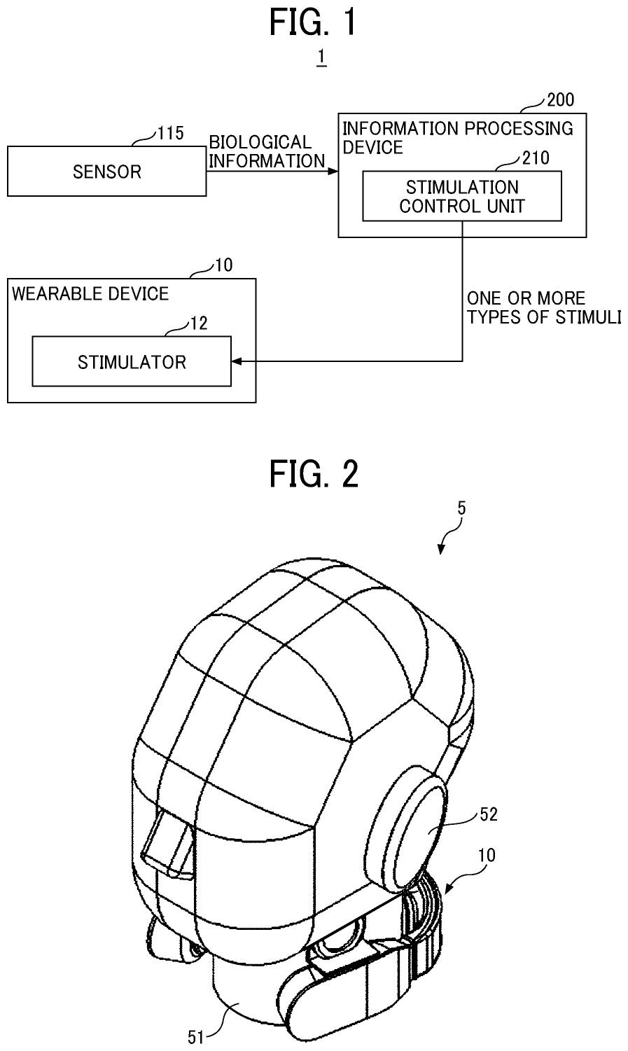

FIG. 1 is a schematic diagram of an arousal state control system 1 according to the present embodiment. An arousal state control system 1 includes a sensor 115, a stimulation control unit 210, and a stimulator 12. The sensor 115 acquires biological information of a user. The stimulation control unit 210 determines one or more types of stimuli to control the state of arousal based on the biological information. The stimulator 12 applies the one or more types of stimuli determined by the stimulation control unit 210 to the user. The stimulator 12 is implemented in the form of a wearable device 10. When the user wears the wearable device 10 and performs work, the user can be stimulated as appropriate for arousal during work.

The wearable device 10, the sensor 115, and an information processing device 200 may be separate devices. Alternatively, at least one of the sensor 115 and the stimulation control unit 210 may be incorporated in the wearable device 10. The information processing device 200 may be connected to the sensor 115 and the wearable device 10 through cables or wireless connection. As the information processing device 200, a microprocessor may be incorporated in the wearable device 10. Alternatively, the information processing device 200 may be, for example, a personal computer, a smartphone, or a tablet terminal independent of the wearable device 10. The sensor 115 may be connected to the wearable device 10 or the information processing device 200 by wiring or wirelessly.

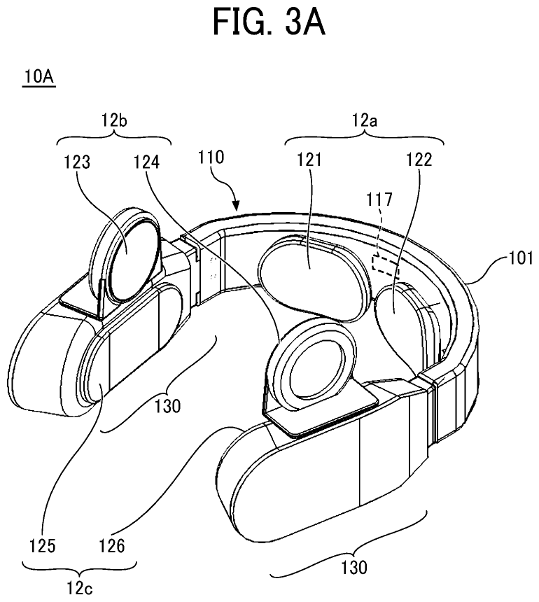

FIG. 2 illustrates the wearable device 10 worn by a user, according to the present embodiment. The wearable device 10 is worn on a body part of a user 5 where the user 5 can be efficiently aroused. The wearable device 10 according to the present embodiment is designed to be worn on a neck 51 of the user 5. Although the wearable device 10 has a U-shape surrounding a part of the periphery of the neck 51 of the user 5 in the example of FIG. 2, the wearable device 10 may have a C-shape. Alternatively, the wearable device 10 may have an O-shape surrounding the entire periphery of the neck 51 of the user 5. Other than such shapes to be worn on the neck 51, the shape and configuration of the wearable device 10 are variable to be worn on a shoulder, a waist, an upper arm, a lower arm, or any other appropriate part of the body of the user 5. The stimulator 12 applies one or more different types of stimuli to the user 5. For example, a stimulus for increasing the level of arousal and a stimulus for decreasing the level of arousal are applied to appropriate parts of the neck 51. A thermal stimulus and an electrical stimulus may be applied simultaneously or alternately to adjust the level of arousal. Three or more types of stimuli may be applied simultaneously or separately. Instead of applying stimuli of different types separately, stimuli of the same type having different strengths may be applied. The level of arousal indicates the degree of arousal or relaxation of a user (target person). In the following description, the level of arousal may be referred to as a state of arousal, whereas the arousal may be referred to as tension or being nervous. In the evaluation of levels of arousal, a high level of arousal indicates that the user is more nervous than at a reference level of arousal, whereas a low level of arousal indicates that the user is more relaxed than at the reference level of arousal. According to the embodiments of the present disclosure, the reference level of arousal is, for example, a “target level of arousal” described later.

In a case where the sensor 115 is incorporated in the wearable device 10, the sensor 115 may be disposed on the inner side of the wearable device 10 at a position where the sensor 115 contacts the carotid artery below an ear 52 of the user 5 or may be worn on an earlobe of the ear 52 or the wrist of the user 5.

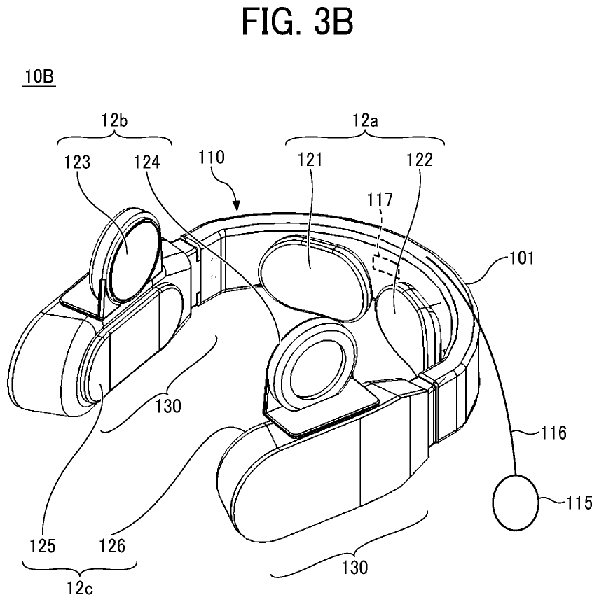

FIGS. 3A, 3B, and 3C illustrate different configurations of the wearable device 10. Specifically, FIG. 3A illustrates a wearable device 10A according to a first example of the present disclosure. The wearable device 10A includes a body 110 and stimulators 12a, 12b, and 12c on the body 110. The stimulators 12a, 12b, and 12c may be referred to simply as stimulator(s) 12 in the following description.

The wearable device 10A illustrated in FIG. 3A is independent of the sensor 115 and the information processing device 200 in the arousal state control system 1 of FIG. 1. The body 110 of the wearable device 10A is provided with a communication device 117. The communication device 117 receives a control signal indicating the result of analysis of biological information from the external information processing device 200. A part or all of the stimulators 12a, 12b, and 12c are driven according to the received control signals. The information processing device 200 may be any device provided that the device has a function of analyzing biological information acquired from the sensor 115 and transmitting the result of analysis to the wearable device 10. As described above, for example, a personal computer, a smartphone, or a tablet terminal may be used as the information processing device 200.

The stimulators 12a, 12b, and 12c generate different stimuli from each other. As an example, the stimulator 12a includes first electrodes 121 and 122 disposed on the inner side of a curved portion 101 of the U-shaped body 110. The first electrodes 121 and 122 come into contact with the back or nape of the neck 51 of the user 5 when the wearable device 10A is worn on the neck 51 of the user 5. The stimulator 12a applies a heat stimulus or a low-frequency electrical stimulus to the nape of the user 5 through the first electrodes 121 and 122. The number of first electrodes is not limited to two. Three or more first electrodes may be disposed along the inner face of the curved portion 101 of the body 110.

When the stimulator 12a generates heat, a heater disposed inside the stimulator 12a is activated according to the control signal received by the communication device 117 to impart heat through the first electrodes 121 and 122. When the stimulator 12a applies the low-frequency electrical stimulus, an oscillation circuit disposed inside the stimulator 12a is turned on to apply low-frequency vibration through the first electrodes 121 and 122. The low-frequency vibration provides electrical muscle stimulation (EMS). The heat or low-frequency vibration applied to the nape of the neck typically serves as a stimulus for relaxation, which relaxes the muscle tension. However, the heat or low-frequency vibration may also serve as a stimulus for arousal depending on individual differences or the stimulus strength.

The stimulator 12b is attached to arms 130 each extending on either side of the curved portion 101 of the body 110. The stimulator 12b includes second electrodes 123 and 124 each projecting from the arm 130 in a width direction of the body 110. When the wearable device 10A is worn on the neck 51 of the user 5, the second electrodes 123 and 124 come into contact with the skin surface around the carotid arteries below the ears 52 or the jaw of the user 5. The stimulator 12b applies a high-frequency electrical stimulus to the carotid arteries of the user 5 through the second electrodes 123 and 124. An oscillation circuit disposed inside the stimulator 12b is turned on to apply high-frequency vibration through the second electrodes 123 and 124. The high-frequency vibration typically serves as a stimulus for relaxation, which improves blood flow. However, the high-frequency vibration may also serve as a stimulus for arousal depending on individual differences or the stimulus strength.

The stimulator 12c is disposed on the inner side of the arms 130 each extending on either side of the curved portion 101 of the body 110. The stimulator 12c includes third electrodes 125 and 126 facing each other. When the wearable device 10A is worn on the neck 51 of the user 5, the third electrodes 125 and 126 come into contact with the skin surface around the carotid arteries along the neck of the user 5. The stimulator 12c applies a cooling stimulus or a heat stimulus to the carotid arteries of the user 5 through the third electrodes 125 and 126. When the stimulator 12c applies the cooling or heat stimulus, the cooling or heat stimulus is generated under the control of ON/OFF of the voltage for driving a Peltier element disposed inside the stimulator 12c and the polarity of the applied voltage. Appropriate cooling and warming of the carotid arteries typically acts in the direction of increasing the level of arousal. However, when the blood vessel is excessively expanded, a cool stimulus, which provides feelings of coolness, may relax the user.

FIG. 3B is a schematic diagram of a wearable device 10B according to a second example of the present disclosure. The wearable device 10B includes the sensor 115 connected to the communication device 117, in addition to the configuration of FIG. 3A. The wearable device 10B illustrated in FIG. 3B includes the sensor 115 and is independent of the information processing device 200 in the arousal state control system 1 of FIG. 1. In the example of FIG. 3B, the sensor 115 is connected to the body 110 through a cable 116 and is electrically connected to the communication device 117. Alternatively, the sensor 115 may be wirelessly connected to the communication device 117. In a case where the sensor 115 can directly communicate with the external information processing device 200 without the communication device 117, the configuration is as illustrated in FIG. 3A. The sensor 115 is a biological sensor such as a heart rate sensor, a pulse sensor, a temperature sensor, or a perspiration sensor. In a case where the sensor 115 is a heart rate sensor or a pulse sensor, the sensor 115 may be a type of sensor attachable to an earlobe or a fingertip of the user 5 or may be another type of sensor attachable to the chest of the user 5.

The communication device 117 transmits the biological information acquired by the sensor 115 to the external information processing device 200. The information processing device 200 analyzes the acquired biological information, determines a stimulus to be applied to the user 5, and transmits the determined stimulus to the wearable device 10B as a control signal. The wearable device 10B drives a part or all of the stimulators 12a, 12b, and 12c, based on the control signal received by the communication device 117. The configurations and functions of the stimulators 12a, 12b, and 12c on the body 110 are as described above with reference to FIG. 3A.

FIG. 3C is a schematic diagram of a wearable device 10C according to a third example of the present disclosure. The wearable device 10C includes a microprocessor 120 in the body 110 and the sensor 115 connected to the microprocessor 120. The wearable device 10C illustrated in FIG. 3C includes the sensor 115 and the information processing device 200 in the arousal state control system 1 of FIG. 1. Although the information processing device 200 is implemented by the microprocessor 120, the information processing device 200 may be implemented by a field programmable gate array (FPGA) or an application-specific integrated circuit (ASIC). In FIG. 3C, the sensor 115 is connected to the body 110 through the cable 116 and is connected to the microprocessor 120. Alternatively, the sensor 115 may be wirelessly connected to the microprocessor 120 with built-in communication functions.

The microprocessor 120 analyzes the biological information acquired from the sensor 115 and drives a part or all of the stimulators 12a, 12b, and 12c on the body 110. The configurations and functions of the stimulators 12a, 12b, and 12c are as described above with reference to FIG. 3A. Other than the configurations illustrated in FIGS. 3A, 3B, and 3C, the body 110 may be simply provided with the microprocessor 120 with wireless communication functions. In this case, the wearable device 10 and the information processing device 200 are integrated and independent of the sensor 115 in the arousal state control system 1 of FIG. 1. The configurations of the arousal state control system 1 and the wearable device 10 may be any configurations provided that the configurations of the arousal state control system 1 and the wearable device 10 allow control of the state of arousal of the user with one or more types of stimuli generated based on biological information acquired.

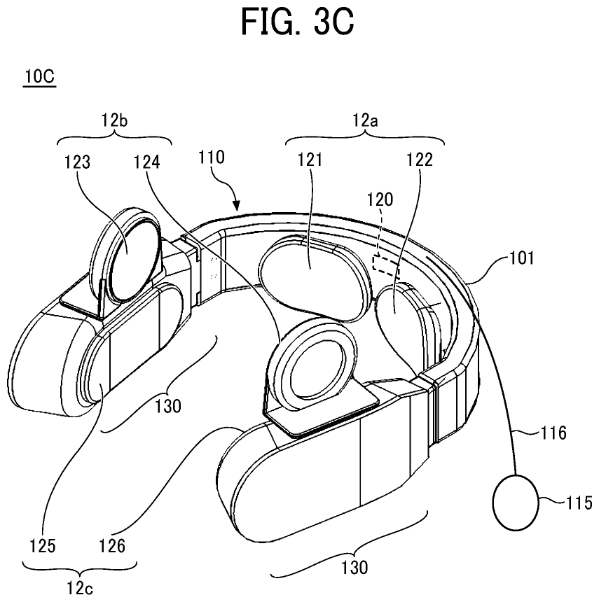

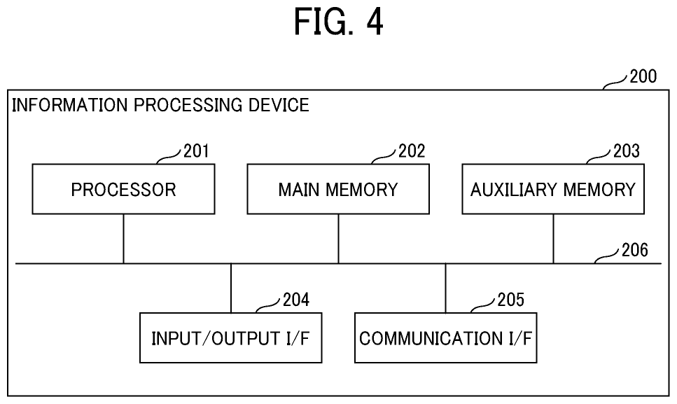

FIG. 4 is a block diagram illustrating a hardware configuration of the information processing device 200 according to the present embodiment. The information processing device 200 includes a processor 201, a main memory 202, an auxiliary memory 203, an input/output interface (I/F) 204, and a communication interface (I/F) 205, which are connected to each other through a system bus 206.

The processor 201, which is implemented by a central processing unit (CPU), executes control processing including various types of arithmetic processing and implements the functions of the stimulation control unit 210. The control processing executed by the processor 201 includes the acquisition of biological information, analysis of the biological information, the determination of a stimulus, the acquisition of a target level of arousal set by the user, and the acquisition of a subjective evaluation input by the user.

The determination of a stimulus includes the determination of, for example, the stimulus type, a combination of stimuli, the duration of stimulation, and the stimulus strength, and correction or change of the determined stimulus type, the determined combination of stimuli, the determined duration of stimulation, and the determined stimulus strength. As will be described later, for example, the type of stimulus to be applied, the duration of stimulation, and the target level of arousal corresponding to work may be corrected based on a change in the difference between the target level of arousal and the level of arousal calculated from the biological information and the subjective evaluation of performance.

The main memory 202 includes a read-only memory (ROM) that stores programs used for the operation of the processor 201 and a random access memory (RAM) that is used as a work area of the processor 201. The auxiliary memory 203 includes a storage device such as a hard disk drive (HDD) or a solid-state drive (SSD). The auxiliary memory 203 stores, in addition to various programs and parameter information to be used to start the programs, information and parameters used for arousal state control.

The input/output interface 204 connects input/output devices such as a display, a touch panel, a speaker, an earphone, a microphone, and a keyboard to the information processing device 200. The communication interface 205 enables communication between the information processing device 200 and an external device through, for example, a public communication network, a local area network (LAN), or a short-range communication standard. The communication between the information processing device 200 and the sensor 115 or the wearable device 10 may be performed according to a short-range wireless communication standard.

A description is given below of a functional configuration of the stimulation control unit 210 according to the present embodiment.

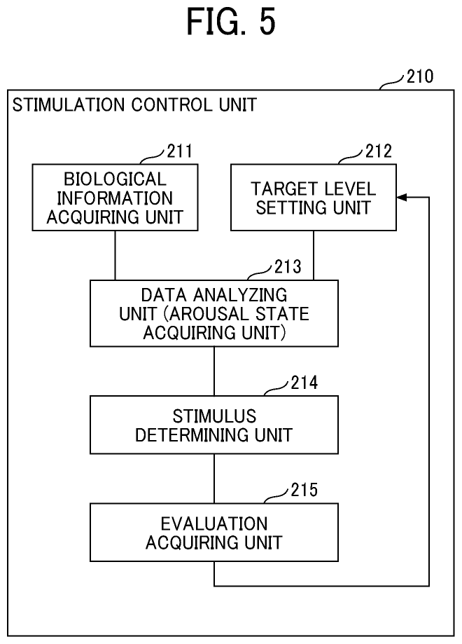

FIG. 5 is a functional block diagram of the stimulation control unit 210 according to the present embodiment. The stimulation control unit 210 is implemented by the processor 201. The stimulation control unit 210 includes a biological information acquiring unit 211, a target level setting unit 212, a data analyzing unit 213, a stimulus determining unit 214, and an evaluation acquiring unit 215.

The biological information acquiring unit 211 acquires biological information of a user who wears the wearable device 10. The biological information may be directly acquired from the sensor 115 worn by the user as described above. Alternatively, the biological information may be acquired from the wearable device 10.

The target level setting unit 212 sets a target level of arousal input by the user. The target level of arousal indicates a target degree of arousal or tension when the user performs work. The target level of arousal may be represented by a numerical value such as a breathing rate, a perspiration level, a heart rate, or the number of blinks. In the present embodiment, a parameter indicating the level of arousal is a ratio (LF/HF) between a low frequency (LF) component and a high frequency (HF) component included in a power spectrum of variation in heart rate, which is obtained by analyzing the heart rate. In this case, the target level of arousal is set as a target LF/HF value.

The LF component is a frequency band in a range of 0.004 hertz (Hz) to 0.150 Hz in the power spectrum of variation in heart rate. The LF component is considered to reflect the activities of both the sympathetic nerves and the parasympathetic nerves. The HF component is a frequency band in a range of 0.150 Hz to 0.400 Hz in the power spectrum of variation in heart rate. The HF component is considered to reflect the activity of parasympathetic nerves. The ratio of LF to HF (i.e., LF/HF) indicates which of the sympathetic nerves and the parasympathetic nerves are dominant to what extent. When the user is aroused or nervous, the sympathetic nerves are dominant over the parasympathetic nerves. In other words, the LF/HF value is relatively large. When the user is relaxed, the parasympathetic nerves are dominant over the sympathetic nerves. In other words, the LF/HF value is relatively small.

The data analyzing unit 213 analyzes the biological information to determine the level of arousal of the user. The data analyzing unit 213 may be referred to as an arousal state acquiring unit in the following description. When the ratio of LF to HF (i.e., LF/HF) is used as a parameter indicating the level of arousal, the data analyzing unit 213 extracts the LF component and the HF component from the acquired spectrum of variation in heart rate data, to calculate the ratio of LF to HF (i.e., LF/HF). The data analyzing unit 213 also refers to the target level of arousal set by the user and specifies the difference between the calculated LF/HF value and the target level of arousal. When the difference between the current LF/HF value and the target level of arousal exceeds an allowable range, the data analyzing unit 213 detects that the user is excessively relaxed or excessively nervous. The data analyzing unit 213 monitors the direction of change in the difference.

The stimulus determining unit 214 determines a stimulus to be applied to the user, based on the result of analysis conducted by the data analyzing unit 213. When the data analyzing unit 213 determines that the user is excessively relaxed, based on the difference between the calculated LF/HF value and the target level of arousal, the stimulus determining unit 214 selects a stimulus for increasing the level of arousal. By contrast, when the data analyzing unit 213 determines that the user is excessively nervous, the stimulus determining unit 214 selects a stimulus for decreasing the level of arousal. Since what type of stimulus is effective for increasing or decreasing the level of arousal varies depending on the user, the tendency of the user may be measured in advance to allow the stimulus determining unit 214 to determine stimuli for arousal and relaxation effective for the user.

For a user who can be relaxed from a nervous state when the carotid arteries of the user are cooled, the stimulus determining unit 214 selects a cool stimulus when the user is excessively nervous. For a user who is aroused by low-frequency electrical stimulation, the stimulus determining unit 214 selects the low-frequency electrical stimulus when the user is excessively relaxed. By contrast, for a user who is aroused from a relaxed state when the carotid arteries of the user are cooled, the stimulus determining unit 214 selects the cool stimulus when the user is excessively relaxed. For a user who is relaxed by low-frequency electrical stimulation, the stimulus determining unit 214 selects the low-frequency electrical stimulus when the user is excessively nervous.

As the first stimulus, a stimulus that is typically considered to be effective for arousal or relaxation may be selected by default. Alternatively, a stimulus may be selected at random from the stimulators 12 of the wearable device 10. By repeatedly acquiring the biological information while the user performs work and determining the state of arousal based on the change in the biological information with respect to the target level of arousal, the stimulation control unit 210 can correct or change the initially selected stimulus as appropriate and lead the level of arousal of the user to the target level of arousal. The correction or change of the stimulus will be described later.

The evaluation acquiring unit 215 acquires an evaluation as to whether the wearable device 10 has improved performance of the user wearing the wearable device 10. The evaluation may be a subjective evaluation that the user inputs to the information processing device 200 after finishing the work. Alternatively, the evaluation may be automatically determined by the data analyzing unit 213 based on the degree of approaching the target level of arousal or the time to reach the target level of arousal.

The stimulation control unit 210 determines different types of stimuli based on the biological information of the user, to control the state of arousal of the user as appropriate. The stimulation control unit 210 corrects or changes, for example, the stimulus type, the duration of stimulation, and the stimulus strength, based on a detected difference between the current level of arousal and the target level of arousal, to lead the level of arousal to an appropriate level of arousal. The feedback on the result of the evaluation of performance allows review of the target level of arousal for each user.

A description is given below of an arousal state control process according to the present embodiment.

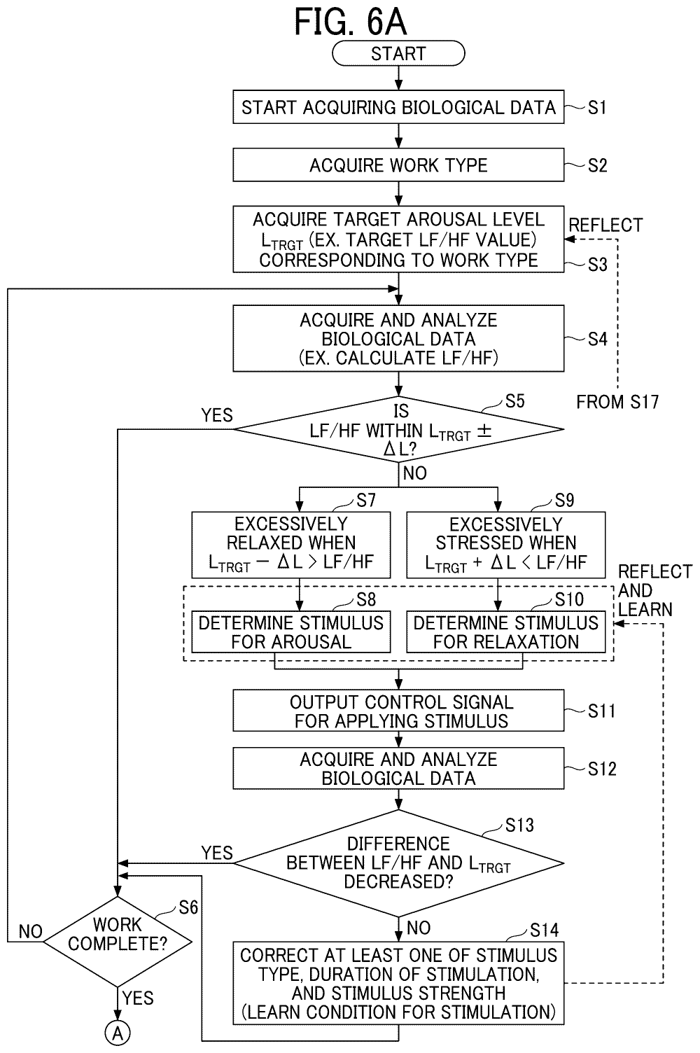

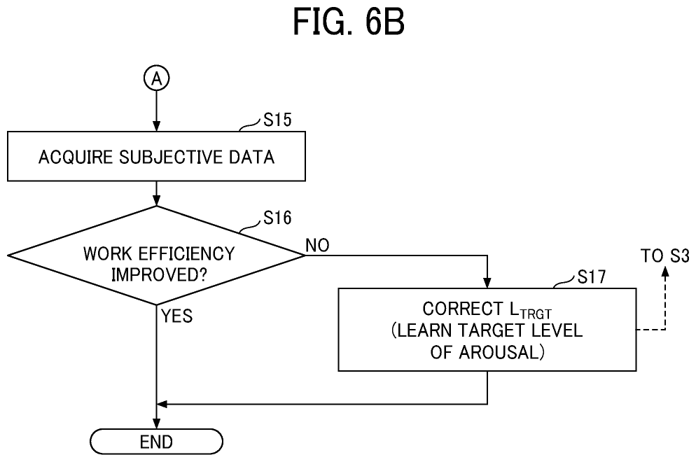

FIG. 6A is a flowchart of arousal state control executed by the stimulation control unit 210. i.e., the processor 201 according to the present embodiment. FIG. 6B is a continuation of the flowchart of FIG. 6A. Specifically, the process illustrated in FIG. 6B continues from a node A illustrated in FIG. 6A. In step S1, the stimulation control unit 210 starts acquiring biological information from the sensor 115. When a user wears the wearable device 10 and activates a predetermined application of the information processing device 200, the stimulation control unit 210 starts acquiring the biological information.

In step S2, the stimulation control unit 210 acquires the work type. In step S3, the stimulation control unit 210 acquires, as a target level of arousal, a target arousal level L_{TRGT} corresponding to the work type. The work type and the target arousal level L_{TRGT} are input to the information processing device 200 by the user. The work type represents the degree of intellectual work in which the thought of the user is involved. The work type is represented by, for example, simple work, normal work, and complicated work. A scheduled work time may be input together with the work type. The target arousal level L_{TRGT} indicates a state of arousal required or desired for the scheduled work.

In the present embodiment, a target LF/HF value as the target level of arousal or a range of target LF/HF values is input by the user. When the ratio of LF to HF (i.e., LF/HF) is used, the target level of arousal may be set in a range not less than 1.5 and not greater than 5.0 as appropriate for the work type. For simple work, the target arousal level L_{TRGT} may be set to be slightly higher so as not to cause drowsiness. For complicated intellectual work, the target arousal level L_{TRGT} may be set slightly lower so that the user is not excessively stressed. The order of steps S2 and S3 may be reversed. Alternatively, the stimulation control unit 210 may simultaneously perform the operations in steps S2 and S3.

In step S4, the stimulation control unit 210 acquires and analyzes the biological data (i.e., biological information). The stimulation control unit 210 analyzes the acquired biological information (e.g., heart rate data) at predetermined time intervals and extracts the LF component and the HF component included in the variation in heart rate data, to calculate the ratio of LF to HF (i.e., LF/HF). For example, the stimulation control unit 210 analyzes the heart rate data for five minutes and calculates the LF/HF value. The processing is updated every minute. However, the present disclosure is not limited to the aforementioned way. The data accumulation time and the time interval for updating may be set as appropriate. For example, the time intervals for data analysis and the calculated LF/HF values are as follows.
Time interval LF/HF
10:00-10:05 2.0
10:01-10:06 1.4
10:02-10:07 1.8
10:03-10:08 1.2 Subsequently, the LF/HF value is calculated for each analysis section, which is a time interval of 5 minutes and shifted every minute.

In step S5, the stimulation control unit 210 determines whether the LF/HF value calculated for each analysis section is within a predetermined range (L_{TRGT}±ΔL) in the vicinity of the target arousal level L_{TRGT}. When the calculated LF/HF value is within the range of L_{TRGT}±ΔL (YES in step S5), the user is in an appropriate state of arousal for the work type. In step S6, the stimulation control unit 210 determines whether the work is complete. When the work is not complete (NO in step S6), the stimulation control unit 210 repeats acquiring and analyzing the biological data (in step S4) and checking the state of arousal (in step S5) until the work is complete (YES in step S6).

When the calculated LF/HF value is not within the range of L_{TRGT}±ΔL (NO in step S5), the state of arousal of the user is away from the target level of arousal set according to the work type. In step S7, the stimulation control unit 210 determines that the user is excessively relaxed for the current work when the calculated LF/HF value is smaller than the allowable range (L_{TRGT}−Δ>calculated value). In this case, in step S8, the stimulation control unit 210 selects a stimulus for increasing the level of arousal, that is, a stimulus for arousal. When the user is effectively aroused by low-frequency electrical stimulation, the stimulator 12a is driven to apply the low-frequency electrical stimulus. The stimulation control unit 210 may determine the duration of stimulation, together with the determination of the stimulus.

In step S9, the stimulation control unit 210 determines that the user is excessively nervous (or stressed) for the current work when the calculated LF/HF value is greater than the allowable range (L_{TRGT}±ΔL<calculated value). In this case, in step S10, the stimulation control unit 210 selects a stimulus for decreasing the level of arousal, that is, a stimulus for relaxation. When the user is relaxed by cool stimulation to the carotid arteries, the stimulator 12c is driven to cool the nape of the user as appropriate. Alternatively, the stimulator 12a may be driven to apply a heat stimulus to the nape of the user to warm the back of the neck. Both the stimulators 12a and 12c may be driven. The stimulation control unit 210 may determine the duration of stimulation and the stimulus strength, together with the determination of the stimulus for relaxation.

In step S11, the stimulation control unit 210 outputs, to the stimulator 12, a control signal for applying the stimulus determined in step S8 or S10. Based on the control signal, the stimulator 12 generates and applies the stimulus.

In step S12, the stimulation control unit 210 continues to acquire and analyze the biological data. In step S13, the stimulation control unit 210 determines whether the difference between the calculated LF/HF value and the target level of arousal is changing in a decreasing direction. When the difference between the calculated LF/HF value and the target level of arousal decreases (YES in step S13), the selected stimulus is appropriate. Thus, the stimulation control unit 210 repeats acquiring and analyzing the biological data (in step S4) and checking the state of arousal (in step S5) until the work is complete (YES in step S6).

By contrast, when the difference between the calculated LF/HF value and the target level of arousal does not decrease (NO in step S13), the selected stimulus is not appropriate. Thus, in step S14, the stimulation control unit 210 corrects or changes at least one of the stimulus type, the duration of stimulation, and the stimulus strength. This correction is reflected in the selection of the stimulus for arousal in step S8 and the selection of the stimulus for relaxation in step S10. Thus, the determination of the stimulus is learned. Subsequently, the stimulation control unit 210 repeats acquiring and analyzing the biological data (in step S4) and checking the state of arousal (in step S5) until the work is complete (YES in step S6).

When the work is complete (YES in step S6), in step S15, the stimulation control unit 210 acquires an evaluation on the improvement of performance. The evaluation may be a subjective evaluation of the user indicating whether the stimulation has improved the user's work efficiency or performance. In step S16, for example, the stimulation control unit 210 determines whether the subjective evaluation indicates an improvement in work efficiency. When the subjective evaluation indicates an improvement in work efficiency (YES in step S16), the stimulation is successful for a specific work type. In this case, the process ends while maintaining the set target level of arousal and the selected stimulus type and duration of stimulation.

By contrast, when the work efficiency is not improved despite the correction of the stimulus (NO in step S16), the target level of arousal set for the specific work type may be inappropriate. In step S17, the stimulation control unit 210 corrects the target arousal level L_{TRGT}. Thus, the process ends. The correction of the target arousal level L_{TRGT} is reflected in the setting of the target arousal level L_{TRGT} in step S3 and learned.

In the arousal state control illustrated in FIGS. 6A and 6B, the stimulation control unit 210 may determine the level of arousal at predetermined time intervals in step S5. For example, the stimulation control unit 210 may determine the state of arousal of the user at intervals of 10 minutes or 15 minutes. With this arousal state control method, the stimulation control unit 210 can control the state of arousal as appropriate for the work performed by the user.

A description is given below of evaluations of arousal state control according to the present embodiment.

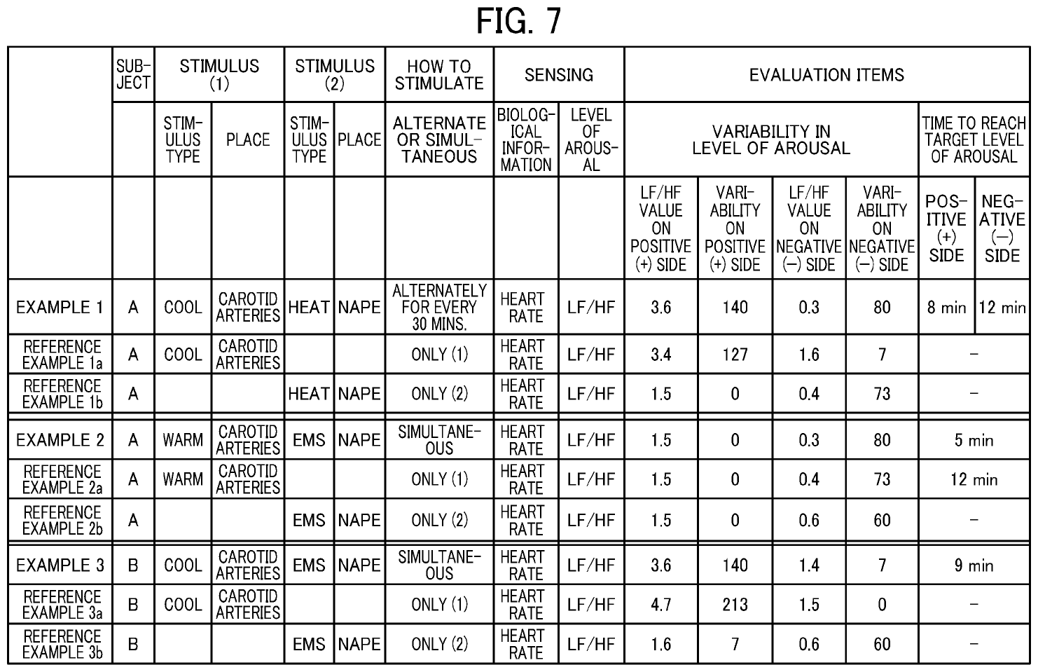

FIG. 7 is a table illustrating the result of evaluations of arousal state control. Each of a user A and a user B wears the wearable device 10. In the evaluations illustrated in FIG. 7, both the users A and B are adult males. The wearable device 10 is applicable to any user regardless of the gender or age of the user. Under the control of the information processing device 200, one or more types of stimuli are applied to each of the users A and B. In all evaluations, the heart rate data is acquired as biological information, whereas the LF/HF value is calculated as a parameter indicating a level of arousal. The LF/HF value is calculated by analyzing the heart rate data for 5 minutes and is updated every minute.

The evaluation items are the variability in the level of arousal and the time to reach the target level of arousal. The variability in the level of arousal indicates variation on the positive (+) side, that is, variation in an overstressed state in which the LF/HF value is higher than the target level of arousal, and variation on the negative (−) side, that is, variation in an overrelaxed state in which the LF/HF value is lower than the target level of arousal. The time to reach the target level of arousal may be also indicated for the positive and negative sides.

A variability R_{CHN} (%), as variability in the level of arousal, is calculated as follows: R_{CHN}=100×(AFT−BFR)/BFR, where BFR represents the LF/HF values for 15 points immediately before stimulation and AFT represents the LF/HF values for 15 points from the maximum LF/HF value during stimulation.

The time to reach the target level of arousal is a time when the average of variabilities of three points reaches a range of 100±10%, which is a target variability. When the target variability is not reached by continuous stimulation for 30 minutes or when the LF/HF value swings upward, the variability R_{CHN} in the level of arousal is evaluated as 0(%).

A description is given below of Example 1 illustrated in FIG. 7.

In Example 1, Stimuli (1) and (2) of different types are applied to the carotid arteries of the user A and the nape of the neck of the user A, respectively. Specifically, Stimulus (1) of Example 1 is a cool stimulus applied to the carotid arteries, whereas Stimulus (2) of Example 1 is a heat stimulus applied to the nape of the neck. The stimulators 12c and 12a of the wearable device 10 are alternately driven to alternately apply Stimulus (1) and Stimulus (2) for 30 minutes each. When Stimulus (1) and Stimulus (2) are alternately applied, the LF/HF value on the positive (+) side (i.e., in the overstressed state) is 3.6, the variability R_{CHN} is 140%, and the time to reach the target level of arousal (i.e., the target LF/HF value) is 8 minutes. The LF/HF value on the negative (−) side (i.e., in the overrelaxed state) is 0.3, whereas the variability R_{CHN} is 80%. The time to reach the target level of arousal is 12 minutes. The user may be aroused more efficiently by different types of stimuli than by a single stimulus. In this case, both the control for increasing the level of arousal and the control for decreasing the level of arousal are achieved. As a result, the target level of arousal is reached in about 10 minutes.

A description is given below of Reference Example 1a illustrated in FIG. 7.

Reference Example 1a is the result of evaluation when Stimulus (1) alone, of the stimuli applied in Example 1, is applied to the carotid arteries. The LF/HF value on the positive (+) side (i.e., in the overstressed state) is 3.4, whereas the variability R_{CHN} is 120%. The LF/HF value on the negative (−) side (i.e., in the overrelaxed state) is 1.6, whereas the variability R_{CHN} is 7%. Reaching the target level of arousal may be difficult when Stimulus (1) alone is continuously applied for 30 minutes. In this example, although cool stimulation to the carotid arteries relaxes the user A to some extent, the target level of arousal is not reached. In other words, the cool stimulus is inappropriate for the user A as a stimulus for increasing the level of arousal. Selecting another stimulus may increase the level of arousal of the user A.

A description is given below of Reference Example 1b illustrated in FIG. 7.

Reference Example 1b is the result of evaluation when Stimulus (2) alone, of the stimuli applied in Example 1, is applied to the nape of the neck. The LF/HF value on the positive (+) side (i.e., in the overstressed state) is 1.4, whereas the variability R_{CHN} is 0%. The LF/HF value on the negative (−) side (i.e., in the overrelaxed state) is 0.4, whereas the variability R_{CHN} is 73%. When Stimulus (2) alone is continuously applied for 30 minutes, the arousal effect is higher as compared with a case where Stimulus (1) is applied. For the user A, the heat stimulation to the nape of the neck increases the level of arousal to some extent. In other words, the heat stimulation can act in the direction of reducing tension.

A description is given below of Example 2 illustrated in FIG. 7.

In Example 2, Stimuli (1) and (2) of different types are applied to the carotid arteries of the user A and the nape of the neck of the user A, respectively. Specifically, Stimulus (1) of Example 2 is a warm stimulus applied to the carotid arteries, whereas Stimulus (2) is (low-frequency) EMS to the nape of the neck. The stimulators 12c and 12a of the wearable device 10 are driven at the same time to apply Stimulus (1) and Stimulus (2) at the same time. When Stimulus (1) and Stimulus (2) are applied at the same time, the LF/HF value on the negative (−) side (i.e., in the overrelaxed state) is 0.3, the variability R_{CHN} is 80%, and the time to reach the target level of arousal is 5 minutes. On the positive (+) side, the LF/HF value is as low as 1.5, and this value hardly fluctuates. The target level of arousal is quickly reached by the application of different stimuli at the same time.

A description is given below of Reference Example 2a illustrated in FIG. 7.

Reference Example 2a is the result of evaluation when Stimulus (1) alone, of the stimuli applied in Example 2, is applied to the carotid arteries. The LF/HF value on the negative (−) side (i.e., in the overrelaxed state) is 0.4, the variability Reux is 73%, and the time to reach the target level of arousal is 12 minutes. The LF/HF value on the positive (+) side is as low as 1.5, and this value hardly fluctuates. Reference Example 2a is included in one of the specific examples of embodiments of the present disclosure. In Reference Example 2a, the level of arousal is controlled toward the target level of arousal by the application of the warm stimulus of Stimulus (1) alone to the carotid arteries. However, it takes more time to reach the target level of arousal than in Example 2 in which the EMS of Stimulus (2) is applied in combination with Stimulus (1).

A description is given below of Reference Example 2b illustrated in FIG. 7.

Reference Example 2b is the result of evaluation when Stimulus (2) alone, of the stimuli applied in Example 2, is applied to the nape of the neck. The LF/HF value on the negative (−) side (i.e., in the overrelaxed state) is 0.6, whereas the variability R_{CHN} is 60%. Although the application of Stimulus (2) alone can increase the level of arousal to some extent, the target level of arousal is not reached by the continuous application of Stimulus (2) for 30 minutes. The LF/HF value on the positive (+) side is as low as 1.5, and this value hardly fluctuates. For this user, the target level of arousal can be achieved more effectively by combining a plurality of types of stimuli than by the EMS alone to the nape of the neck.

A description is given below of Example 3 illustrated in FIG. 7.

In Example 3, Stimuli (1) and (2) of different types are applied to the carotid arteries of the user B and the nape of the neck of the user B, respectively. Specifically, Stimulus (1) of Example 3 is the cool stimulus applied to the carotid arteries, whereas Stimulus (2) is the (low-frequency) EMS to the nape of the neck. The stimulators 12c and 12a of the wearable device 10 are driven at the same time to apply Stimulus (1) and Stimulus (2) at the same time. When Stimulus (1) and Stimulus (2) are applied at the same time, the LF/HF value on the positive (+) side (i.e., in the overstressed state) is 3.6, whereas the variability R_{CHN} is 140%. The LF/HF value on the negative (−) side (i.e., in the overrelaxed state) is 1.4, whereas the variability R_{CHN} is 7%. The time to reach the target level of arousal is 9 minutes. A slight fluctuation of the LF/HF value on the negative (−) side may indicate that Stimulus (1) acts on relaxation while Stimulus (2) finely adjusts the level of arousal in the direction of slightly increasing the level of arousal. Application of different stimuli enables both the control in the direction of decreasing the level of arousal and the control in the direction of increasing the level of arousal. As a result, the target level of arousal is easily reached.

A description is given below of Reference Example 3a illustrated in FIG. 7.

Reference Example 3a is the result of evaluation when the cool stimulus of Stimulus (1) alone, of the stimuli applied in Example 3, is applied to the carotid arteries. The LF/HF value on the positive (+) side (i.e., in the overstressed state) is 4.7, whereas the variability R_{CHN} is 213%. The LF/HF value on the negative (−) side (i.e., in the overrelaxed state) is 1.4, whereas the variability R_{CHN} is 0%. For the user B, the strength of Stimulus (1) is too strong and the LF/HF value swings upward, resulting in a failure of evaluation. In this case, by combining Stimulus (2) in the direction of increasing the level of arousal with Stimulus (1) as in Example 3, the level of arousal can be controlled to a desired level of arousal.

A description is given below of Reference Example 3b illustrated in FIG. 7.

Reference Example 3b is the result of evaluation when Stimulus (2) alone, of the stimuli applied in Example 3, is applied to the nape of the neck. The LF/HF value on the negative (−) side (i.e., in the overrelaxed state) is 0.6, whereas the variability RCIN is 60%. The target level of arousal is not reached by the continuous application of Stimulus (2) alone for 30 minutes. The LF/HF value on the positive (+) side is as low as 1.5, and this value hardly fluctuates. For this user, the target level of arousal can be achieved more effectively by combining a plurality of types of stimuli than by the EMS alone to the nape of the neck.

The result illustrated in FIG. 7 indicates that the same stimulus can be a stimulus for relaxation or a stimulus for arousal, depending on the user. The tendency of the user may be acquired in advance to classify multiple types of stimuli that can be applied by the wearable device 10 into a stimulus for relaxation or a stimulus for arousal and record the classified multiple types of stimuli in a built-in memory of the information processing device 200 or the microprocessor 120. When a high relaxation effect or an appropriate arousal effect can be obtained by combining two or more different types of stimuli, the combination of different types of stimuli may be recorded in the built-in memory of the information processing device 200 or the microprocessor 120. For example, when the relaxation effect or the arousal effect is excessive with a single application of the warm stimulus to the carotid arteries or the low-frequency electrical stimulus to the nape of the neck, simultaneous application of the warm stimulus to the carotid arteries and the low-frequency electrical stimulus to the nape of the neck can finely adjust the state of arousal to a desired state of arousal.

When the arousal state control method according to the embodiments of the present disclosure is implemented by a computer program, an arousal state control program is installed in the information processing device 200. The arousal state control program causes the processor 201 to execute: a procedure (a) of acquiring biological information of a user; a procedure (b) of analyzing the acquired biological information and determining one or more types of stimuli to control a state of arousal of the user; and a procedure (c) of notifying a stimulator of the determined one or more types of stimuli.

A description is given below of several examples of the position and configuration of stimulators.

Initially, a description is given below of several configuration examples of the stimulators 12a, 12b, and 12c. The stimulators 12a, 12b, and 12c are designed to reduce interference with the body of the user wearing the wearable device 10 as much as possible so that the user feels comfortable when wearing the wearable device 10.

A description is given below of a first configuration example.

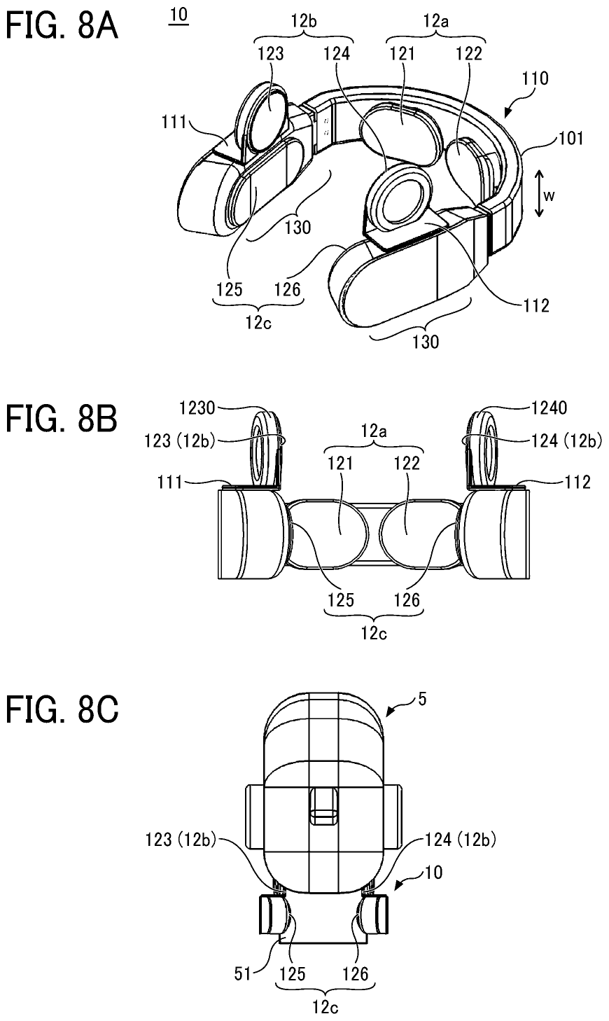

FIGS. 8A to 8C illustrate the first configuration example of the stimulators 12. Specifically, FIG. 8A is a perspective view of the wearable device 10. FIG. 8B is a front view of the wearable device 10. FIG. 8C is a front view of the wearable device 10 worn by the user 5. The body 110 of the wearable device 10 is provided with the stimulators 12a, 12b, and 12c. The stimulators 12a and 12c are positioned in a circumferential direction on the inner side of the body 110. The stimulators 12b and 12c are aligned in a direction intersecting the circumferential direction of the body 110, for example, in a direction parallel to the width (w) direction of the body 110.

When the user 5 wears the wearable device 10, the first electrodes 121 and 122 of the stimulator 12a come into contact with the back or nape of the neck 51 to apply a heat stimulus or a low-frequency electrical stimulus. On the other hand, the stimulator 12c comes into contact with the sides of the neck 51 where the carotid arteries run, to apply a cool stimulus or a warm stimulus with the third electrodes 125 and 126. Combining different types of stimuli effectively leads the user to a target state of arousal.

The stimulator 12b is held by holders 111 at the edges of the arms 130 at both sides of the curved portion 101 and projects in the width (w) direction of the body 110. The stimulator 12b applies a high-frequency electrical stimulus to the sides of the neck 51 of the user 5 wearing the wearable device 10. Applying different types of stimuli to the same part near the carotid arteries with the stimulators 12b and 12c effectively leads the user to a target state of arousal.

The same effect can be obtained by using the stimulators 12a and 12b at the same time.

Depending on the body shape of the user 5, the upper end of the stimulator 12b may contact the jawline. As illustrated in FIG. 8B, the stimulator 12b has a circular contour and the outer circumferences of frames 1230 and 1240 holding the second electrodes 123 and 124, respectively, are gently curved surfaces. Thus, the stimulator 12b can reduce interference with the jawline of the user.

A description is given below of a second configuration example.

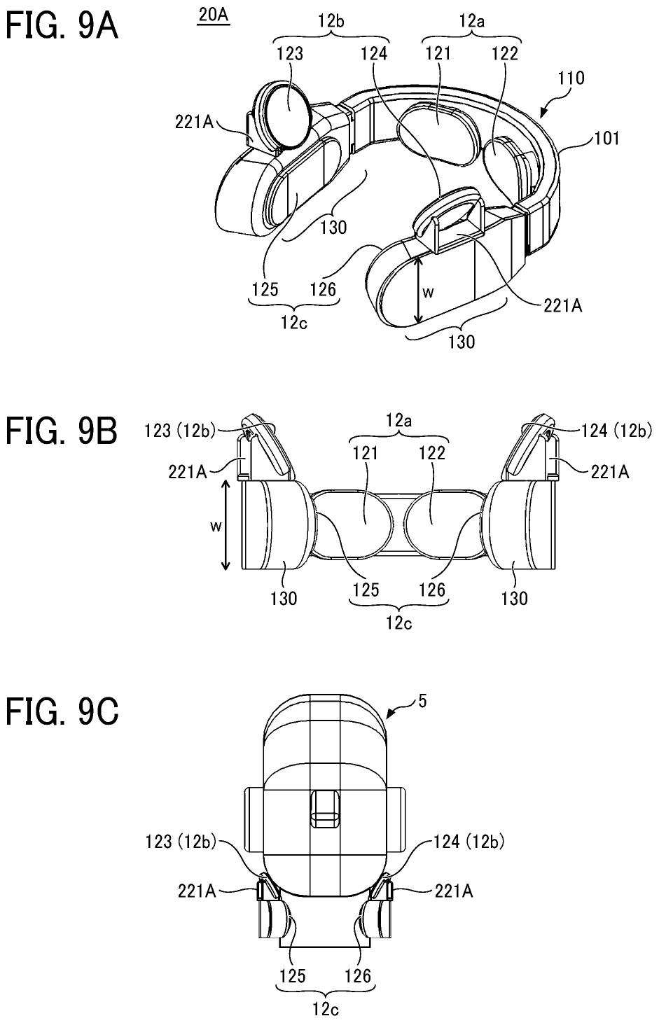

FIGS. 9A to 9C illustrate the second configuration example of the stimulators 12. Specifically, FIG. 9A is a perspective view of a wearable device 20A. FIG. 9B is a front view of the wearable device 20A. FIG. 9C is a front view of the wearable device 20A worn by the user 5. The body 110 of the wearable device 20A is provided with the stimulators 12a, 12b, and 12c. The position and configuration of the stimulators 12a and 12c are the same as those of the stimulators 12a and 12c of the wearable device 10 illustrated in FIGS. 8A to 8C.

The stimulator 12b is held by holders 221A such that the second electrodes 123 and 124 are inclined outward relative to the width (w) direction of the body 110 (more specifically, the arm 130). Since the stimulator 12b is inclined with respect to the width (w) direction of the body 110, the stimulator 12b is also inclined with respect to the third electrodes 125 and 126 of the stimulator 12c positioned inside the arms 130. The stimulator 12b inclined relative to the width (w) direction of the body 110 causes the second electrodes 123 and 124 to fit the contour of the user 5, to effectively stimulate the carotid arteries. In addition, the stimulator 12b that does not dig into the jawline of the user 5 enhances the comfortableness for the user 5 wearing the wearable device 20A.

A description is given below of a third configuration example.

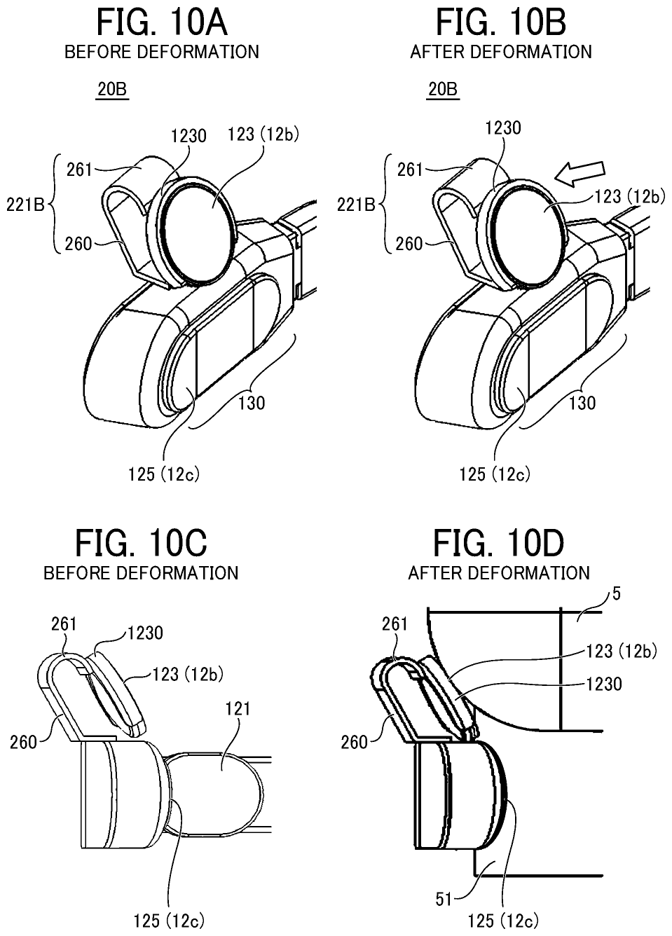

FIGS. 10A to 10D illustrate the third configuration example of the stimulators 12. The stimulator 12b of a wearable device 20B illustrated in FIGS. 10A to 10D is held by holders 221B such that the stimulator 12b can be displaced. The position and configuration of the stimulators 12a and 12c are the same as those of the stimulators 12a and 12c of the wearable device 10 illustrated in FIGS. 8A to 8C and the stimulators 12a and 12c of the wearable device 20A illustrated in FIGS. 9A to 9C.

Specifically, FIG. 10A illustrates the stimulator 12b, which is attached to the arm 130 of the wearable device 20B, before being displaced (deformed). FIG. 10B illustrates the stimulator 12b after being displaced (deformed). FIG. 10C is a partial front view of the wearable device 20B before the stimulator 12b is displaced (deformed). FIG. 10D is a front view of the wearable device 20B worn by the user 5 after the stimulator 12b is displaced (deformed). The stimulator 12b is held on the arm 130 by the flexible holders 221B. The stimulator 12c is positioned inside the arms 130.

Each of the holders 221B is made of an elastic material such as resin and has a first portion 260 fixed to the arm 130 and a second portion 261 curved and extending from an end opposite to a fixed end of the first portion 260 to hold the stimulator 12b. The second portion 261 may be coupled to the frame 1230 holding the second electrode 123 of the stimulator 12b. As illustrated in FIG. 10B, when pressure is applied to the second electrode 123 of the stimulator 12b, the holder 221B is deformed to change the inclination angle of the stimulator 12b with respect to the arm 130.

When the user 5 wears the wearable device 20B and the jawline of the user 5 comes into contact with the second electrode 123 while applying pressure to the second electrode 123, the second electrode 123 is inclined in a direction away from the jaw (under the car) of the user 5 as illustrated in FIG. 10D. Such an inclination causes the stimulator 12b to fit the contour of the face of the user 5 and prevents the stimulator 12b from digging into the jawline of the user 5.

The holders 221B may be made of any material provided that the holders 221B can be deformed by pressure to displace the electrode faces of the stimulator 12b. For example, metallic plate springs may be used as the holders 221B. Like the second electrode 123, the second electrode 124 facing the second electrode 123 of the stimulator 12b is held by the deformable holder 221B. When the jawline of the user 5 comes into contact with the second electrode 124 while applying pressure to the second electrode 124, the second electrode 124 is displaced in a direction away from the jaw of the user 5. The wearable device 20B having the configuration illustrated in FIGS. 10A to 10D leads the user 5 to a target state of arousal with a combination of different types of stimuli. The stimulator 12b of the wearable device 20B conforming to the shape of the neck or the jaw of the user 5 enhances the comfortableness for the user 5 wearing the wearable device 20B.

A description is given below of a fourth configuration example.

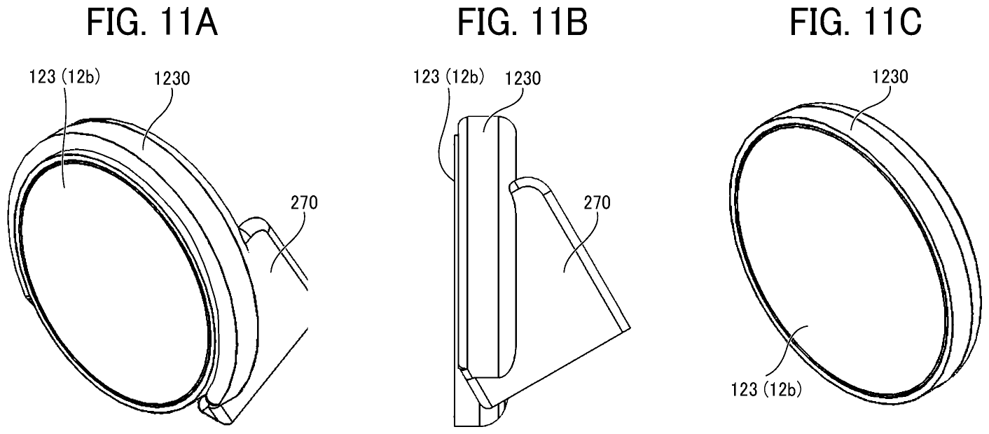

FIGS. 11A to 11C illustrate the fourth configuration example of the stimulators 12. The stimulator 12b is attachable to and detachable from the body 110. Specifically, FIG. 11A is a perspective view of the stimulator 12b held by a holder 270. FIG. 11B is a side view of the stimulator 12b. FIG. 11C is a schematic view of the stimulator 12b detached. The position and configuration of the stimulators 12a and 12c are the same as those of the stimulators 12a and 12c of the wearable device 10 illustrated in FIGS. 8A to 8C, the stimulators 12a and 12c of the wearable device 20A illustrated in FIGS. 9A to 9C, and the stimulators 12a and 12c of the wearable device 20B illustrated in FIGS. 10A to 10D. The stimulator 12b may be attachable to and detachable from the body 110 (see FIG. 9A) while being held by the holder 270. Alternatively, the stimulator 12b with the frame 1230 may be attachable to and detachable from the holder 270 that is fixed to the arm 130 of the body 110.

In a case where the stimulator 12b is attachable to and detachable from the body 110 together with the holder 270, the holder 270 may be fitted into a desired one of a plurality of recesses formed in the circumferential direction of the body 110 or the arm 130. The recesses may have a stopper function. Since the user 5 can change the position of the stimulator 12b to a desired position according to the body shape, the stimulator 12b can effectively stimulate the carotid arteries of the user 5. In addition, cleaning and maintenance of the stimulator 12b is facilitated.

In a case where the stimulator 12b is attachable to and detachable from the holder 270, a stimulator that applies a different type of stimulus can be attached to the holder 270. For example, another stimulator 12 may be attached to the holder 270 to generate vibration for generating music, far-infrared rays, or aroma, instead of a high-frequency electrical stimulus. In addition, cleaning and maintenance of the stimulator 12b is facilitated. The configuration illustrated in FIGS. 11A to 11C leads the user 5 to a target state of arousal with a combination of different types of stimuli.

A description is given below of a fifth configuration example.

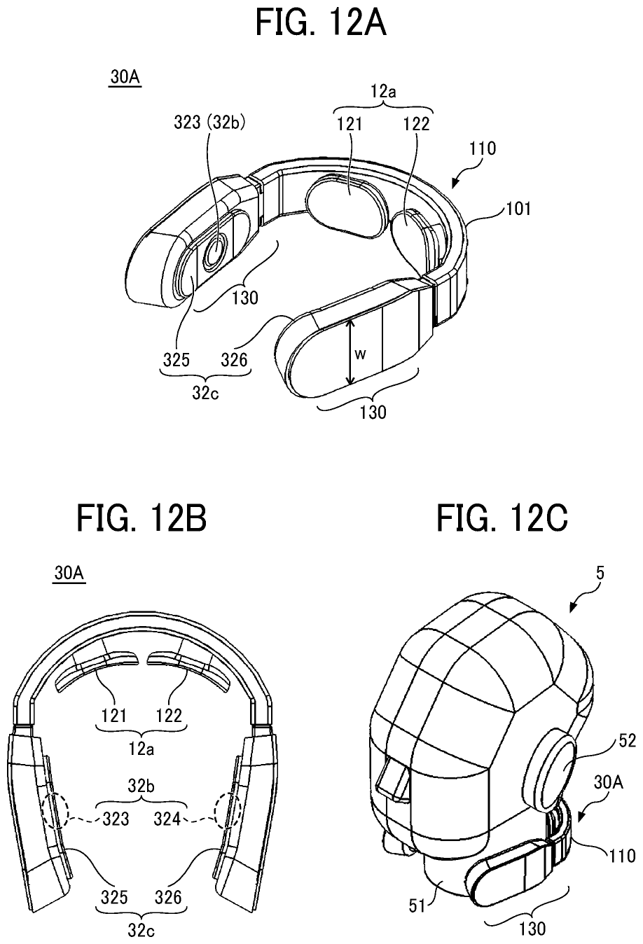

Each of FIGS. 12A to 12C illustrates a wearable device 30A, as the fifth configuration example. Specifically, FIG. 12A is a perspective view of the wearable device 30A. FIG. 12B is a top view of the wearable device 30A. FIG. 12C is a perspective view of the wearable device 30A worn by the user 5. The wearable device 30A includes the stimulator 12a, a stimulator 32b, and a stimulator 32c on the body 110. The stimulators 12a, 32b, and 32c are positioned in the circumferential direction on the inner side of the U-shaped body 110. The stimulator 32b and the stimulator 32c are disposed at the same position. Second electrodes 323 and 324 of the stimulator 32b are surrounded by third electrodes 325 and 326 of the stimulator 32c, respectively, in common planes of the stimulator 32b and the stimulator 32c. The position and configuration of the stimulator 12a are the same as those illustrated in FIG. 8A to FIG. 11C.

In this example, each of the third electrodes 325 and 326 has an elliptical planar shape to keep a relatively wide contact face that contacts the user 5. However, the shape of the third electrodes 325 and 326 is not limited to the elliptical planar shape. Alternatively, for example, each of the third electrodes 325 and 326 may have an elliptical shape or a rectangular shape. Each of the third electrodes 325 and 326 has an opening near the center. The second electrodes 323 and 324 of the stimulator 32b are positioned in the openings of the third electrodes 325 and 326, respectively. The surfaces of the second electrodes 323 and 324 are substantially aligned with the surfaces of the third electrodes 325 and 326, respectively. The second electrode 323 and the third electrode 325 are electrically insulated from each other by, for example, an air layer or an insulating resin embedded between the second electrode 323 and the third electrode 325. Similarly, the second electrode 324 and the third electrode 326 are electrically insulated from each other by, for example, an air layer or an insulating resin embedded between the second electrode 324 and the third electrode 326.

The second electrodes 323 and 324 of the stimulator 32b may apply a high-frequency electrical stimulus to the carotid arteries, whereas the third electrodes 325 and 326 of the stimulator 32c may apply a cool stimulus or a warm stimulus to the same positions of the carotid arteries. Since the stimulators 32b and 32c are individually controlled, different types of stimuli can be applied to the same position of the neck simultaneously, alternately, or at an appropriate time.

The configuration illustrated in FIGS. 12A to 12C includes no portion projecting from the body 110 or the arm 130 in the width (w) direction of the body 110 or the arm 130. Such a simple design enhances the comfortableness for the user 5 wearing the wearable device 30A. With this configuration, the wearable device 30A can exert an effect similar to the effect exerted in the above configurations by applying different types of stimuli at desired times.

A description is given below of a sixth configuration example.

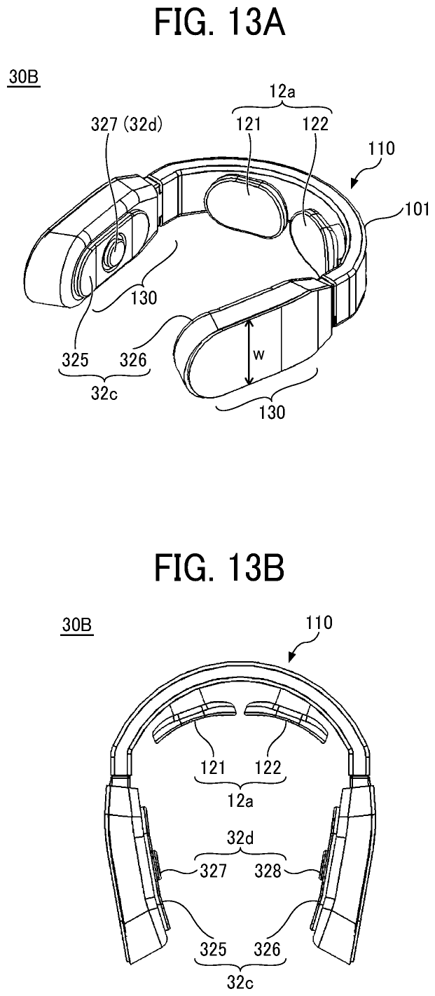

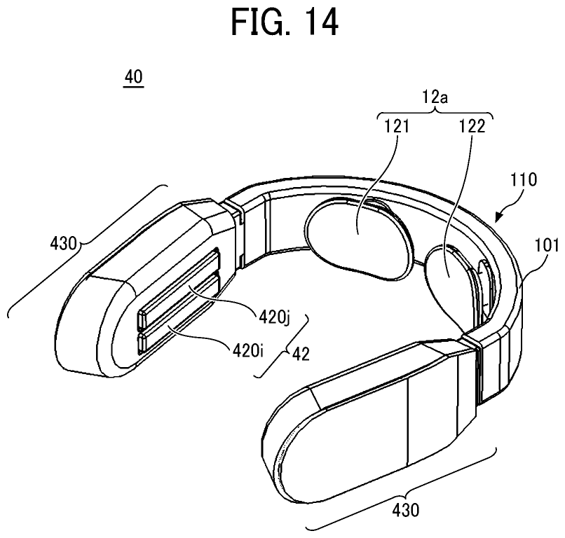

Each of FIGS. 13A and 13B illustrates a wearable device 30B, as the sixth configuration example. Specifically, FIG. 13A is a perspective view of the wearable device 30B. FIG. 13B is a top view of the wearable device 30B. The wearable device 30B includes the stimulator 12a, the stimulator 32c, and a stimulator 32d on the body 110. The stimulators 12a, 32c, and 32d are positioned in the circumferential direction on the inner side of the U-shaped body 110. The position and configuration of the stimulators 12a and 32c are the same as those illustrated in FIGS. 12A to 12C.

The stimulator 32d is surrounded by the stimulator 32c in a common plane of the stimulator 32c and the stimulator 32d. When the wearable device 30B is not worn, fourth electrodes 327 and 328 of the stimulator 32d project from the surfaces of the third electrodes 325 and 326 of the stimulator 32c, respectively, toward the inside of the body 110 as illustrated in FIG. 13B. When the wearable device 30B is worn by the user 5, the fourth electrodes 327 and 328 come into contact with the sides of the neck 51 of the user 5 (see FIG. 12C) and are retracted into the arms 130. As a result, the surfaces of the fourth electrodes 327 and 328 are aligned with the surfaces of the third electrodes 325 and 326, respectively. In other words, the perspective view of the wearable device 30B worn by the user 5 is the same as the perspective view of the wearable device 30A of FIG. 12C.

The configuration illustrated in FIGS. 13A and 13B includes no portion projecting from the body 110 or the arm 130 in the width (w) direction of the body 110 or the arm 130. Such a simple design enhances the comfortableness for the user 5 wearing the wearable device 30B. In addition, the fourth electrodes 327 and 328 of the stimulator 32d retractably projecting in a direction orthogonal to the inner faces of the arms 130 ensure contact with the user 5 when the user 5 wears the wearable device 30B, and effectively stimulate the neck 51 of the user 5. With this configuration, the wearable device 30B can exert an effect similar to the effect exerted in the above configurations by applying different types of stimuli at desired times.

A description is given below of a seventh configuration example.

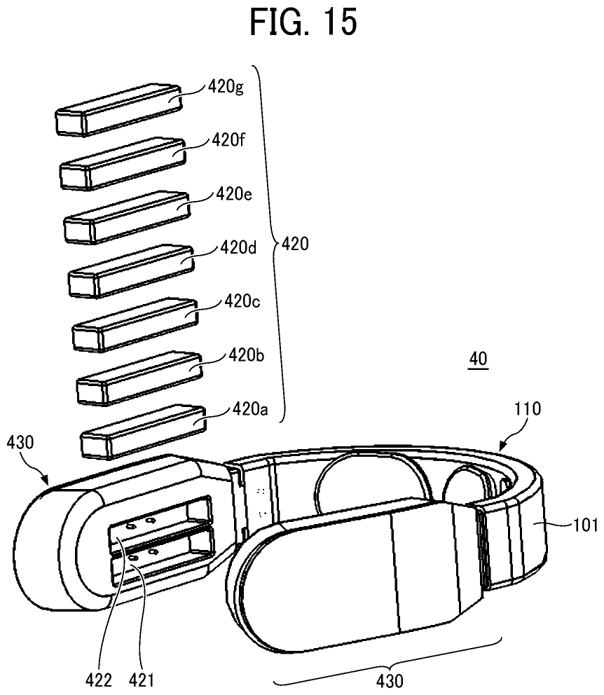

FIG. 14 illustrates a wearable device 40, as the seventh configuration example. According to the seventh configuration example, the stimulators are attachable to and detachable from the body 110 of the wearable device 40. Like the first to sixth configuration examples, the wearable device 40 applies a plurality of types of stimuli. The wearable device 40 includes, for example, the stimulator 12a and a stimulator 42 on the body 110. The position and configuration of the stimulator 12a are the same as those illustrated in FIGS. 12A to 12C. The stimulator 42 is disposed inside an arm 430. For example, the arm 430 is rotatable in a plane orthogonal to a longitudinal axis of the body 110. Different types of stimulation components can be replaceably or detachably set on the arm 430.

The stimulator 12a applies a low-frequency electrical stimulus with the first electrodes 121 and 122 that are disposed at the center of a nape-contact portion of the body 110. The nape-contact portion of the body 110 contacts the nape of the neck of the user 5. The arm 430 at each end of the body 110 is provided with the stimulator 42 to which one or more types of stimulation components can be detachably set. For example, stimulation components 420j and 420i are positioned vertically in parallel in the stimulator 42. Specifically, the stimulation component 420j that applies a cool stimulus by cooling is positioned above the stimulation component 420i that applies a warm stimulus by heating. The stimulation components 420i and 420j come into contact with the vicinity of the carotid artery to selectively or alternately apply the warm stimulus and the cool stimulus, respectively.

FIG. 15 illustrates the wearable device 40 and stimulation components 420 detached from the body 110 of the wearable device 40. Inside the arm 430 of the wearable device 40 are sockets 421 and 422 that receive the stimulation components 420 such that the stimulation components 420 can be detached from the sockets 421 and 422. Each of the sockets 421 and 422 includes, on the bottom face, an electrode pad that is electrically connected to one of the stimulation components 420. The stimulator 42 includes desired ones of the stimulation components 420 fitted into the sockets 421 and 422.

As the stimulation components 420, a plurality of types of chips shaped to fit into the sockets 421 and 422 are prepared to apply predetermined stimuli. The stimulation components 420 include stimulation components 420a to 420g. Specifically, the stimulation component 420a applies a low-frequency electrical stimulus. The stimulation component 420b applies a high-frequency electrical stimulus. The stimulation component 420c applies a cool stimulus by cooling and a warm stimulus by heating. The stimulation component 420d applies a stimulus by vibration. The stimulation component 420e applies an acoustic stimulus with sound. The stimulation component 420f applies a light stimulus by illumination. The stimulation component 420g applies an aroma stimulus with aroma. A wearer of the wearable device 40 can select a desired stimulus depending on, for example, the physical condition or mood of the wearer and set the stimulation components 420 in an attachment/detachment portion of each of the sockets 421 and 422, to use the wearable device 40.

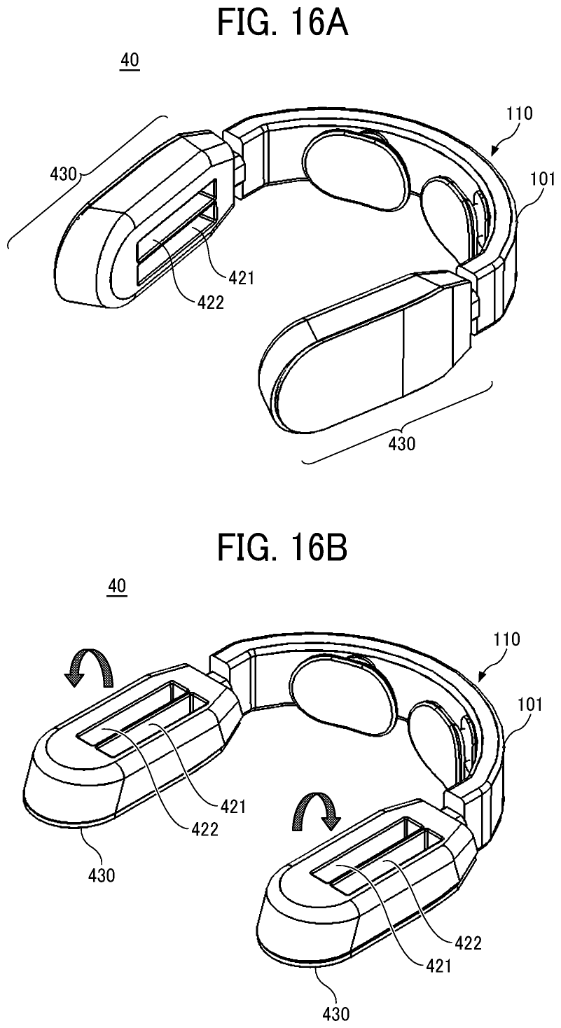

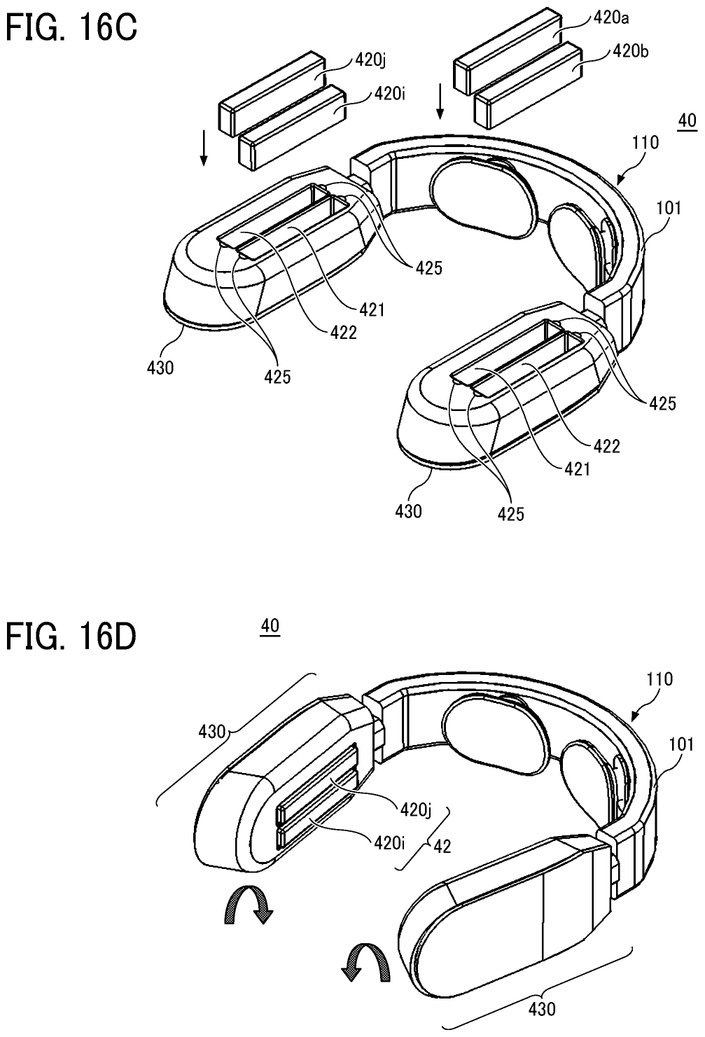

FIGS. 16A to 16D are diagrams illustrating several steps of attaching the stimulation components 420 to the wearable device 40 according to the present embodiment. FIG. 16A illustrates the wearable device 40 without the stimulation components 420 set on the body 110. The stimulation components 420 are not set in the sockets 421 and 422 inside the arm 430 at each end of the body 110.

The arms 430 are rotated in the directions indicated by arrows in FIG. 16B, with respect to the longitudinal axis of the body 110 curved in an arch shape, so that the surface provided with the sockets 421 and 422 faces upward to facilitate the insertion and removal of the stimulation components 420. The bottom face of each of the sockets 421 and 422 may be provided with a latch spring to facilitate the removal of the stimulation component 420, together with the electrode pad to be electrically connected to the stimulation component 420.

In FIG. 16C, desired ones of the stimulation components 420 are inserted into the sockets 421 and 422. The same type of stimulation components 420 may or may not be arranged at the corresponding positions between the left and right arms 430. The stimulation components 420 selected for the left arm 430 may be different in type from the stimulation components 420 selected for the right arm 430. Specifically, for example, the stimulation components 420i and 420j may be set on the right arm 430, whereas the stimulation components 420a and 420b may be set on the left arm 430. Note that the right arm 430 is the arm 430 on the right side of the user 5 wearing the wearable device 40, whereas the left arm 430 is the arm 430 on the left side of the user 5 wearing the wearable device 40. As a removal mechanism in each of the sockets 421 and 422, a holding mechanism using a magnetic force or a notch 425 may be disposed at each edge of the sockets 421 and 422 as illustrated in FIG. 16C, instead of the latch spring described above with reference to FIG. 16B. In the example of FIG. 16C, the notch 425 is located at the center of each of the opposed short sides of each of the sockets 421 and 422. However, the location of the notch 425 is not limited to the location illustrated in FIG. 16C. Alternatively, for example, the notch 425 may be located at any appropriate position along the opening of each of the sockets 421 and 422.

The arms 430 are rotated with respect to the body 110 in the directions indicated by arrows in FIG. 16D with the stimulation components 420 set on the body 110 so that the surfaces on which the stimulation components 420 are set face inward.

The wearable device 40 is ready to be worn, with the inner face of each of the arms 430 provided with the stimulator 42.

According to the seventh configuration example, the position of the arms 430 can be moved to facilitate the operation of the arms 430 to attach or detach the stimulation components 420. Thus, the user-friendliness is enhanced for the attaching and detaching operations of the stimulation components 420. Since the surface of the arm 430 into which the stimulation components 420 are inserted can be directed upward, maintenance such as cleaning is facilitated. As described above, the arms 430 are rotatable about the axis with respect to the body 110. In addition, the arms 430 may be movable in a direction along the axis.

Accordingly, when the wearable device 40 is worn on the neck, the relative positions of the stimulator 12a (see FIG. 14) for the nape of the neck and the stimulator 42 for the carotid arteries can be adjusted. Since the stimulators can be moved to positions to conform to the neck of the wearer, the wearable device 40 can stimulate the wearer more reliably as compared with a wearable device including fixed stimulators. Since the wearable device 40 has no portion projecting upward from the arms 430, the wearable device 40 does not bite into the jaw of the wearer at the time of use. Optionally, each of the arms 430 may be provided with three or more sockets to allow more types of stimulation components 420 to be attached, depending on the size of the arms 430 and the size of sockets (i.e., the size of the stimulation components 420).

Since the level of response to the stimulus varies depending on the user, the wearable device 40 that allows the selection of stimuli suitable for the user can stimulate the user more effectively than typical wearable devices including stimulators. As the period of use increases, the level of response may decrease due to the habituation of the body to stimulation. However, according to the present configuration example, the replacement of the stimulation component 420 with another one resets the habituation by new stimulation and increases the level of response. Since the stimulation components 420 can be selectively combined, various models do not have to be developed. In case of failure of the stimulation component 420, the stimulation component 420 can be repaired or replaced alone. When a new stimulus is developed, the stimulation component 420 can be purchased alone.

The stimulus type of each of the stimulation components 420 may be identifiable. The stimulus type can be easily identified by, for example, indicating characters or markings, or color coding. The user may be notified of a mismatch between the setting on the body of the wearable device and the type of the stimulator or the stimulus component, if any, so that the user can replace the type of the stimulator in advance. To secure operability at the time of attaching and detaching the stimulation components 420, the stimulation components 420 may have a bilaterally symmetrical or vertically symmetrical outer-diameter shape to be attached in either direction. A cylindrical outer diameter of the stimulation components 420 and a cylindrical shape of the sockets 421 and 422 may allow the user to attach the stimulation components 420 to the sockets 421 and 422 without worrying about the angle of attachment. In a configuration that limits the positions at which the stimulation components 420 are attached with respect to the body 110 of the wearable device 40, a reverse attachment prevention mechanism using the magnetic south and north poles or an incompatibility mechanism using a mechanical shape may be provided. Accordingly, the stimulation components 420 can be reliably attached to the body 110. When the stimulation components 420 are not attached to the sockets 421 and 422, the sockets 421 and 422 may be covered with a dummy cover to ensure design.

A description is given of several example combinations of stimuli.

Table 1 illustrates several example combinations of stimuli for the wearable device 40. The stimulators 12a and 42 illustrated in FIG. 14 are used. The stimulator 42 on each of the arms 430 can apply two types of stimuli.

TABLE 1

| | CAROTID | CAROTID | | | CAROTID | CAROTID |
|---|---|---|---|---|---|---|
| | ARTERY | ARTERY | | | ARTERY | ARTERY |
| | UPPER LEFT | LOWER LEFT | NAPE LEFT | NAPE RIGHT | LOWER RIGHT | UPPER RIGHT |
| EXAMPLE 4 | HIGH | COOLING | LOW | LOW | COOLING | HIGH |
| | FREQUENCY | & HEATING | FREQUENCY | FREQUENCY | & HEATING | FREQUENCY |
| EXAMPLE 4-1 | COOLING | HIGH | LOW | LOW | HIGH | COOLING |
| | & HEATING | FREQUENCY | FREQUENCY | FREQUENCY | FREQUENCY | & HEATING |
| EXAMPLE 4-2 | LOW | LOW | LOW | LOW | LOW | LOW |
| | FREQUENCY | FREQUENCY | FREQUENCY | FREQUENCY | FREQUENCY | FREQUENCY |
| EXAMPLE 4-3 | HIGH | COOLING | LOW | LOW | HIGH | COOLING |
| | FREQUENCY | & HEATING | FREQUENCY | FREQUENCY | FREQUENCY | & HEATING |
| EXAMPLE 4-4 | VIBRATION | SOUND | HIGH | LOW | LIGHT | SMELL |
| | | | FREQUENCY | FREQUENCY | | |

Example 4 indicates, for example, the default stimuli of the wearable device 40. Specifically, the stimulator 12a applies a low-frequency electrical stimulus to the left and right of the nape of the neck, whereas the stimulator 42 applies a high-frequency electrical stimulus to the upper sides of the left and right carotid arteries and cool/warm stimuli to the lower sides of the left and right carotid arteries. The stimuli can be freely selected or changed from such default stimuli. In Example 4-1, the stimuli applied to the upper and lower sides of the carotid arteries are exchanged. Some users may prefer to receive a cool stimulus by cooling and a warm stimulus by heating at a position close to the faces of the users. In this case, the combination in Example 4-1 is preferable.

In Example 4-2, all the six stimuli applied by the stimulators 12a and 42 indicate the same low-frequency electrical stimulus. Such arrangement is possible for users who prefer low-frequency electrical stimulation. In Example 4-3, the stimulation components 420 positioned on the upper and lower sides of the carotid arteries are different between the left and right sides of the wearable device 40. Thus, the wearable device 40 provides asymmetric stimulation. For users who strongly feel discomfort with bilaterally symmetrical stimuli, the stimulation components 420 can be positioned to provide asymmetrical stimulation. Example 4-4 indicates a combination of six different stimuli applied by the stimulators 12a and 42. For users who seek more types of stimuli, a combination as in Example 4-4 can be provided.

Asymmetric stimulation can be achieved by selecting the stimulation components 420 different between the left and right or upper and lower sides of the wearable device 40. For example, some users may be more effectively aroused by stimuli different between the left and right: a high-frequency electrical stimulus alone on the right and a low-frequency electrical stimulus alone on the left.

Although the arousal state control and the configuration of the wearable device have been described above based on the specific embodiments or examples of the present disclosure, various modifications may be made within the scope of the technical idea of the present invention. In a case where the sensor 115, the communication device 117, or the microprocessor 120 is incorporated in the wearable device 10, the sensor 115, the communication device 117, or the microprocessor 120 may be connected to or disposed at an appropriate position of the arm 130, instead of being connected to or disposed at the curved portion 101 of the body 110. The biological information acquired by the sensor 115 is not limited to heart rate data. Other biological data such as a breathing rate, a sweating state, or the number of blinks may be detected to determine whether the user is nervous or relaxed. In addition to or instead of the thermal stimulus and the electrical stimulus, an aroma stimulus, an acoustic stimulus, a light stimulus, or a combination of these stimuli may be applied. Although the wearable device 10 to be worn on the neck as illustrated in FIG. 2 does not interfere with the work of the user and is effective in controlling the state of arousal, the wearable device of one or more embodiments of the present disclosure may be a wearable device to be worn on the back of the shoulder or the forearm.

The first to seventh configuration examples may be combined with each other. For example, the wearable device 40 may further include the stimulator 12b having any configuration of the first to third configuration examples described above. In the seventh configuration example, in a case where a part of the stimulation component 420 inserted into the socket projects from a contact face that contacts the carotid arteries due to, for example, the influence of variations of components, a retraction mechanism may be provided to retract a projecting portion of the stimulation component 420 to the contact face as in the sixth configuration example (FIGS. 13A and 13B). The retraction mechanism may be, for example, an elastic member made of resin or a metal spring. The retraction mechanism may have any configuration provided that the stimulation component 420 can be retracted by a certain external pressure.

A description is given below of wearable devices according to other examples of the present disclosure.

As described above, the wearable device of one or more embodiments of the present disclosure is not limited to a wearable device to be worn on the neck. The wearable device may be attached to any part of the body other than the neck, such as a wrist, an earlobe, a fingertip, a forearm, a thigh, or the back of a shoulder, provided that biological information such as a pulse, a body temperature, or a sweating state can be measured and a stimulus can be applied to the part.

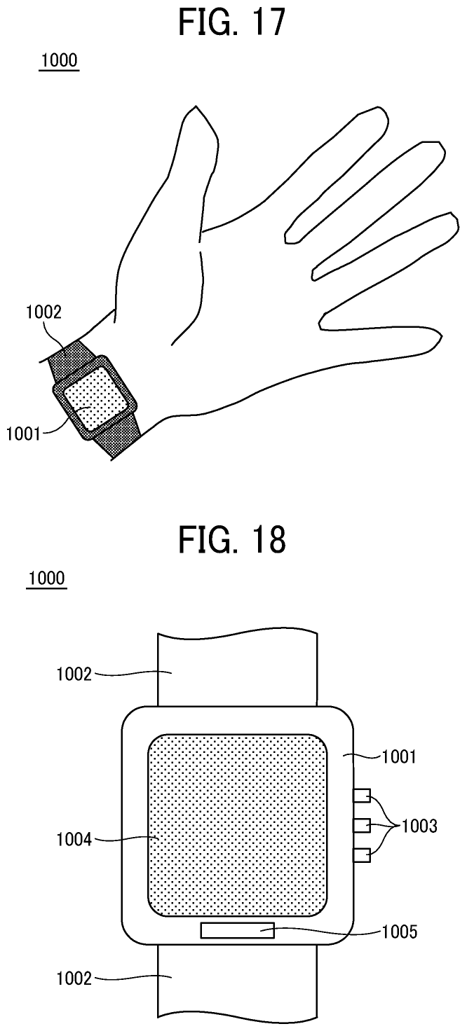

FIG. 17 is a diagram of a wearable device 1000 according to another example of the present disclosure. FIG. 18 is a schematic diagram illustrating a configuration of a skin-contact side of the wearable device 1000 of FIG. 17. The skin-contact side of the wearable device 1000 contacts the skin of a user. The wearable device 1000 can be worn on a wrist. The wearable device 1000 includes a body 1001 and an attaching unit 1002. As illustrated in FIG. 18, the body 1001 includes an operation unit 1003, a stimulator 1004, and a sensor 1005. The stimulator 1004 is positioned to contact, for example, the inner side of a wrist. The sensor 1005 is disposed at a position where biological information can be easily acquired, for example, at the inner side of the wrist or at a position where the sensor 1005 can contact an artery. The operation unit 1003 may be disposed on a side of the body 1001. For example, the operation unit 1003 allows the user to turn on or off the power and adjust the stimulus strength. The communication device 117 (see FIGS. 3A and 3B) or the microprocessor 120 (scc FIG. 3C) is incorporated in the body 1001 to transmit the acquired biological information to a remote processor or analyze the acquired biological information inside the wearable device 1000. Although FIG. 18 illustrates the single stimulator 1004, the number of stimulators is not limited to one. For example, another stimulator may be disposed on the same surface of the body 1001 on which the stimulator 1004 is disposed, to apply a plurality of types of stimuli such as a thermal stimulus and an electrical stimulus.

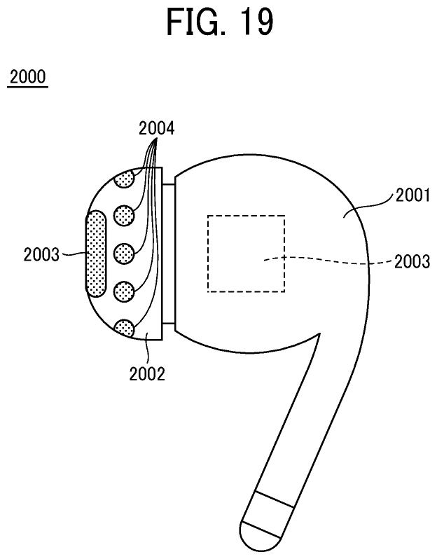

FIG. 19 is a diagram of a wearable device 2000 according to yet another example of the present disclosure. The wearable device 2000 can be worn on an ear. The wearable device 2000 includes a body 2001, an attaching unit 2002, a stimulator 2003, and a sensor 2004. The body 2001 incorporates the communication device 117 or the microprocessor 120.

The wearable device 2000 may incorporate a speaker as the stimulator 2003. The speaker can apply a sound stimulus to the user. The attaching unit 2002 has an earlobe-contact-face through which the attaching unit 2002 contacts an earlobe. For example, in a case where the stimulator 2003 applies a thermal stimulus, the stimulator 2003 is preferably positioned on the earlobe-contact-face of the attaching unit 2002 to easily contact the ear. Similarly, the sensor 2004 is preferably disposed at a position on the attaching unit 2002 where the sensor 2004 can easily contact the ear (earlobe).

Each of the wearable devices 1000 and 2000 can apply stimuli of different types or different strengths to a part of the user in contact with the corresponding wearable device, based on the biological information of the user.

A description is given below of several aspects of the present disclosure.

According to a first aspect, a wearable device to be worn by a user includes a body and a plurality of stimulators on the body. The plurality of stimulators applies, based on biological information of the user, stimuli of different types or different strengths to the user.

According to a second aspect, in the wearable device of the first aspect, the plurality of stimulators generates and applies a first stimulus that increases a level of arousal of the user and a second stimulus that decreases the level of arousal of the user.

According to a third aspect, in the wearable device of the first aspect, the plurality of stimulators includes a first stimulator and a second stimulator. The first stimulator is disposed at a first portion of the body to generate a first stimulus. The second stimulator is disposed at a second portion of the body to generate a second stimulus different from the first stimulus. The first stimulator and the second stimulator are aligned in a circumferential direction of the body or in a direction intersecting the circumferential direction.

According to a fourth aspect, in the wearable device of the third aspect, the body includes a curved portion and a pair of arms extending from both ends of the curved portion. The first stimulator is disposed on the curved portion of the body to apply the first stimulus to a first portion of the user. The second stimulator is disposed on the pair of arms to apply the second stimulus to a second portion of the user.

According to a fifth aspect, in the wearable device of the third aspect, the first stimulator is disposed on an inner face of the body. The second stimulator projects from the body in a width direction of the body.

According to a sixth aspect, the wearable device of the fifth aspect further includes a holder that holds the second stimulator to incline a stimulus applying face of the second stimulator with respect to the width direction of the body. The stimulus applying face is a face through which the second stimulator applies the second stimulus.

According to a seventh aspect, in the wearable device of the sixth aspect, the holder is deformable to adjust an angle of the stimulus applying face with respect to the body.

According to an eighth aspect, in the wearable device of the sixth aspect, the holder detachably holds the second stimulator.

According to a ninth aspect, in the wearable device of the third aspect, the second stimulator is surrounded by the first stimulator.

According to a tenth aspect, in the wearable device of the ninth aspect, the second stimulator projects from a surface of the first stimulator and is retractable to a position of the surface of the first stimulator.

According to an eleventh aspect, the wearable device of any one of the first to tenth aspects further includes a sensor that detects the biological information.

According to a twelfth aspect, the wearable device of any one of the first to tenth aspects further includes a processor that analyzes the biological information and determines the stimuli of different types based on a result of analysis of the biological information.

According to a thirteenth aspect, the wearable device of any one of the first to tenth aspects further includes a communication device that transmits the biological information to an external information processing device external to the wearable device and that receives, from the external information processing device, a control signal to drive the plurality of stimulators.

According to a fourteenth aspect, in the wearable device of the first aspect, the plurality of stimulators includes at least one stimulator that applies variable types of stimuli.

According to a fifteenth aspect, in the wearable device of the fourteenth aspect, the plurality of stimulators applies the stimuli of different types or different strengths asymmetrically to the user.

According to a sixteenth aspect, the wearable device of the fourteenth aspect further includes an arm movable with respect to the body at each end of the body. Stimulation components are detachably disposed on the arm to apply the stimuli of different types.

According to a seventeenth aspect, an arousal state control system includes a sensor, a stimulation control unit, and a wearable device. The sensor acquires biological information of a user. The stimulation control unit determines a plurality of types of stimuli to control a state of arousal based on the biological information. The wearable device applies the plurality of types of stimuli determined by the stimulation control unit to the user. The wearable device includes a body and a stimulator on the body to apply the plurality of types of stimuli.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention. Any one of the above-described operations may be performed in various other ways, for example, in an order different from the one described above.

The present invention can be implemented in any convenient form, for example using dedicated hardware, or a mixture of dedicated hardware and software. The present invention may be implemented as computer software implemented by one or more networked processing apparatuses. The processing apparatuses include any suitably programmed apparatuses such as a general purpose computer, a personal digital assistant, a Wireless Application Protocol (WAP) or third-generation (3G)-compliant mobile telephone, and so on. Since the present invention can be implemented as software, each and every aspect of the present invention thus encompasses computer software implementable on a programmable device. The computer software can be provided to the programmable device using any conventional carrier medium (carrier means). The carrier medium includes a transient carrier medium such as an electrical, optical, microwave, acoustic or radio frequency signal carrying the computer code. An example of such a transient medium is a Transmission Control Protocol/Internet Protocol (TCP/IP) signal carrying computer code over an IP network, such as the Internet. The carrier medium may also include a storage medium for storing processor readable code such as a floppy disk, a hard disk, a compact disc read-only memory (CD-ROM), a magnetic tape device, or a solid state memory device.

The functionality of the elements disclosed herein may be implemented using circuitry or processing circuitry which includes general purpose processors, special purpose processors, integrated circuits, application specific integrated circuits (ASICs), digital signal processors (DSPs), field programmable gate arrays (FPGAs), conventional circuitry and/or combinations thereof which are configured or programmed to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein or otherwise known which is programmed or configured to carry out the recited functionality. When the hardware is a processor which may be considered a type of circuitry, the circuitry, means, or units are a combination of hardware and software, the software being used to configure the hardware and/or processor.

This patent application is based on and claims priority to Japanese Patent Application Nos. 2022-192274, filed on Nov. 30, 2022, and 2023-173617, filed on Oct. 5, 2023, in the Japan Patent Office, the entire disclosure of each of which is hereby incorporated by reference herein.

### REFERENCE SIGNS LIST

1 Arousal state control system
10, 10A, 10B, 10C, 30, 30A, 40, 1000, 2000 Wearable device
12, 12a, 12b, 12c. 32b, 32c. 32d, 42, 1004, 2003 Stimulator
101 Curved portion
110, 1001, 2001 Body
111, 221A, 221B, 270 Holder
115, 1005, 2004 Sensor
116 Cable
117 Communication device
120 Microprocessor
121, 122 First electrode
123, 124, 323, 324, 423, 424 Second electrode
125, 126, 325, 326 Third electrode
130 Arm
200 Information processing device
201 Processor
202 Main memory
203 Auxiliary memory
204 Input/output interface
205 Communication interface
210 Stimulation control unit
211 Biological information acquiring unit
212 Target level setting unit
213 Data analyzing unit
214 Stimulus determining unit
215 Evaluation acquiring unit
221A, 221B, 270 Holder
420a to 420g, 420i, 420j Stimulation component
1002, 2002 Attaching unit
1003 Operation unit

1. A wearable device to be worn by a user, the wearable device comprising:
a body; and
a plurality of stimulators on the body to apply, based on biological information of the user, stimuli of different types or different strengths to the user.

2. The wearable device according to claim 1,
wherein the plurality of stimulators is to generate and apply, as the stimuli, a first stimulus that increases a level of arousal of the user and a second stimulus that decreases the level of arousal of the user.

3. The wearable device according to claim 1,
wherein the plurality of stimulators includes:
a first stimulator at a first portion of the body to generate a first stimulus of the stimuli; and
a second stimulator at a second portion of the body to generate a second stimulus of the stimuli, the second stimulus being different from the first stimulus,
and
wherein the first stimulator and the second stimulator are aligned in a circumferential direction of the body or in a direction intersecting the circumferential direction.

4. The wearable device according to claim 3,
wherein the body includes:
a curved portion; and
a pair of arms extending from both ends of the curved portion,
wherein the first stimulator is disposed on the curved portion of the body to apply the first stimulus to a first portion of the user, and
wherein the second stimulator is disposed on the pair of arms to apply the second stimulus to a second portion of the user.

5. The wearable device according to claim 3,
wherein the first stimulator is disposed on an inner face of the body, and
wherein the second stimulator projects from the body in a width direction of the body.

6. The wearable device according to claim 5, further comprising:
a holder holding the second stimulator to incline a stimulus applying face of the second stimulator with respect to the width direction of the body, the stimulus applying face being a face through which the second stimulator is to apply the second stimulus.

7. The wearable device according to claim 6,
wherein the holder is deformable to adjust an angle of the stimulus applying face with respect to the body.

8. The wearable device according to claim 6,
wherein the holder detachably holds the second stimulator.

9. The wearable device according to claim 3,
wherein the second stimulator is surrounded by the first stimulator.

10. The wearable device according to claim 9,
wherein the second stimulator projects from a surface of the first stimulator and is retractable to a position of the surface of the first stimulator.

11. The wearable device according to claim 1, further comprising:
a sensor to detect the biological information.

12. The wearable device according to claim 1, further comprising:
a processor configured to analyze the biological information and determine the stimuli of different types based on a result of analysis of the biological information.

13. The wearable device according to claim 1, further comprising communication circuitry configured to:
transmit the biological information to an external information processing device external to the wearable device; and
receive, from the external information processing device, a control signal to drive the plurality of stimulators.

14. The wearable device according to claim 1,
wherein the plurality of stimulators includes at least one stimulator to apply variable types of stimuli.

15. The wearable device according to claim 14,
wherein the plurality of stimulators is to apply the stimuli of different types or different strengths asymmetrically to the user.

16. The wearable device according to claim 14, further comprising:
an arm movable with respect to the body at each end of the body, to detachably receive stimulation components to apply the stimuli of different types.

17. An arousal state control system comprising:
a sensor to acquire biological information of a user;
stimulation control circuitry configured to determine a plurality of types of stimuli to control a state of arousal of the user based on the biological information; and
a wearable device to apply the plurality of types of stimuli determined by the stimulation control unit to the user,
the wearable device including:
a body; and
a stimulator on the body to apply the plurality of types of stimuli.
